(19) United States
(12) Patent Application Publication
(10) Pub. No.: US 2025/0169739 A1
(43) Pub. Date: May 29, 2025

(54) RECONSTRUCTION OF BRAIN ELECTRICAL ACTIVITY USING SPATIALLY RESOLVED ELECTROENCEPHALOGRAPHY

(71) Applicant: The Regents of the University of California, Oakland, CA (US)

(72) Inventors: Lawrence Robert Frank, San Diego, CA (US); Vitaly Ludwig Galinsky, La Jolla, CA (US)

(21) Appl. No.: 18/961,288

(22) Filed: Nov. 26, 2024

Related U.S. Application Data
(60) Provisional application No. 63/604,009, filed on Nov. 29, 2023.

(51) Int. Cl. A61B 5/372; A61B 5/00; A61B 5/055

(52) U.S. Cl. CPC A61B 5/372; A61B 5/0042; A61B 5/055

(57) ABSTRACT

Methods, systems, and devices are described for reconstructing spatially resolved electrical activity in the brain. In some example embodiments, EEG and MRI data are used to estimate volumetric distribution of electrostatic potential inside the MRI domain throughout the entire brain. Spatially and temporally varying field estimates can be generated using a brain wave model which is based on weakly evanescent transverse cortical wave propagation and constrained using the tissue properties gained from the MRI data. The disclosed techniques enable brain activity imaging with high spatial and temporal resolution, thereby providing a tool for assessing functional brain states and monitoring changes in those states in relation to various normal and pathological conditions.

### CROSS-REFERENCE TO RELATED APPLICATION

This patent document claims priority to and benefits of U.S. Provisional Application No. 63/604,009, entitled “SPATIALLY RESOLVED ELECTROENCEPHALOGRAPHY CONSTRAINED WITH TISSUE PROPERTIES BY REGULARIZED ENTROPY,” and filed on Nov. 29, 2023. The entire content of the above noted patent application is incorporated by reference as part of the disclosure of this patent document.

### TECHNICAL FIELD

This patent document is generally related to techniques to reconstruct the spatial and temporal distribution of electric fields in the brain using electroencephalography (EEG) data.

### BACKGROUND

The ability to image the dynamic electrical activity inside brain tissues has profound implications for the understanding of cognition. Direct imaging of the electric field networks in the human brain from EEG data has potential for immediate benefit to a broad range of important scientific and clinical questions concerning brain electrical activity, while also providing an inexpensive and portable alternative to functional magnetic resonance imaging (fMRI).

### SUMMARY

The disclosed embodiments, among other features and benefits, relate to methods and systems to reconstruct spatially resolved electrical activity in the brain from EEG data. The disclosed embodiments enable brain activity imaging with high spatial and temporal resolution, without the concomitant distortions found in fMRI. Additionally, the disclosed embodiments provide a tool for assessing functional brain states and monitoring changes in those states in relation to various normal and pathological conditions. Thus, the disclosed embodiments enable quantification and analysis across multiple conditions, facilitating objective evaluation of brain function in both typical and atypical presentations.

The disclosed embodiments use EEG and MRI data (e.g., high resolution anatomical, diffusion MRI, or functional MRI) to estimate volumetric distribution of electrostatic potential inside the MRI domain throughout the entire brain. In the current state of the art, there exists no technique that can spatially resolve the electrical fields in the brain from EEG data.

In some implementations, the disclosed embodiments use EEG electrical signals gathered from multiple electrodes placed on the scalp of a human subject, in conjunction with MRI brain tissue data, to estimate the volumetric distribution of electric field potential throughout the brain. Spatially and temporally varying field estimates are generated by modeling the propagation of weakly evanescent transverse cortical waves. Wave propagation under this model is further constrained using the tissue properties gained from the MRI data. The disclosed embodiments can be used to reconstruct spatially resolved brain activations in single or multiple human subjects.

In one aspect, a method for determining volumetric distribution of electric field potential within a brain is disclosed. The method comprises: acquiring at least two datasets including electroencephalography (EEG) data associated with a volume of the brain and magnetic resonance imaging (MRI) data associated with the volume of the brain; determining, using an approximation for a volumetric distribution of electrostatic potential in an anisotropic and inhomogeneous medium, a frequency-dependent electrostatic field potential that is based on the EEG data and tissue properties of the brain estimated from the MRI data, the tissue properties including morphological and electrical properties at locations within the volume of the brain; iteratively constructing an approximate solution for the frequency-dependent electrostatic field potential within the volume of the brain using a brain wave model constrained by the tissue properties and based on weakly evanescent transverse cortical brain wave propagation; determining spatiotemporal modes of electrical activity within the volume of the brain by solving the approximation using the approximate solution; and obtaining the volumetric distribution of the electric field potential within the volume of the brain based on the spatiotemporal modes.

In another aspect, a method for reconstructing electric field potential within a volume of a brain is disclosed. The method comprises: acquiring at least two datasets associated with the volume of the brain, the at least two datasets including electroencephalography (EEG) data and magnetic resonance imaging (MRI) data; determining a frequency-dependent electrostatic field potential that is based on the EEG data and tissue properties of the brain estimated from the MRI data, the tissue properties including frequency-dependent electrical properties within the volume of the brain; determining, based in-part on the frequency-dependent electrostatic field potential and entropy field decomposition analysis, spatiotemporal modes of electrical activity within the volume of the brain; determining a distribution of the electric field potential within the volume of the brain based on spatiotemporal modes of electrical activity within the volume of the brain; and obtaining, based on the distribution, a reconstructed image of the brain describing spatiotemporal patterns of the electrical activity within the volume of the brain.

In yet another aspect, a device comprising a processor and a memory with instructions stored thereon is disclosed. In some implementations, the instructions upon execution by the processor cause the processor to perform a method for determining volumetric distribution of electric field potential within a brain. In some implementations, the method comprises: acquiring at least two datasets including electroencephalography (EEG) data associated with a volume of the brain and magnetic resonance imaging (MRI) data associated with the volume of the brain; determining, using an approximation for a volumetric distribution of electrostatic potential in an anisotropic and inhomogeneous medium, a frequency-dependent electrostatic field potential that is based on the EEG data and tissue properties of the brain estimated from the MRI data, the tissue properties including morphological and electrical properties at locations within the volume of the brain; iteratively constructing an approximate solution for the frequency-dependent electrostatic field potential within the volume of the brain using a brain wave model constrained by the tissue properties and based on weakly evanescent transverse cortical brain wave propagation; determining spatiotemporal modes of electrical activity within the volume of the brain by solving the approximation using the approximate solution; and obtaining the volumetric distribution of the electric field potential within the volume of the brain based on the spatiotemporal modes.

### BRIEF DESCRIPTION OF THE DRAWINGS

This application contains at least one drawing executed in color. Copies of this application with color drawing(s) will be provided by the Office upon request and payment of the necessary fees.

FIG. 1 shows example differences in SPatially resolved EEG Constrained with Tissue properties by Regularized Entropy (SPECTRE) power modes obtained in an example use case based on the disclosed embodiments.

FIG. 2 shows an example of a power difference atlas obtained in an example use case based on the disclosed embodiments.

FIG. 3A shows an example of statistical significance between SPECTRE power modes obtained in an example use case based on the disclosed embodiments.

FIG. 3B shows an example of statistical significance between the difference in SPECTRE power modes obtained in an example use case based on the disclosed embodiments.

FIG. 4 shows an example of space-time information trajectories and associated connectivity eigenmodes obtained in an example use case based on the disclosed embodiments.

FIG. 5 shows example differences in space-time information trajectories obtained in an example use case based on the disclosed embodiments.

FIG. 6 shows an example comparison of reconstructed fMRI activity with SPECTRE reconstructed EEG data obtained in an example use case based on the disclosed technology.

FIG. 7 shows an example comparison of reconstructed fMRI activity with SPECTRE reconstructed EEG data obtained in an example use case based on the disclosed technology.

FIG. 8A-F show example results obtained in an example use case based on the disclosed technology.

FIG. 9 show example results obtained in an example use case based on the disclosed technology.

FIG. 10 shows a comparison of activation maps derived from fMRI to activation maps derived from EEG in accordance with implementations of the disclosed technology.

FIG. 11 shows an example of a reconstructed sensor configuration obtained in an example use case based on the disclosed technology.

FIG. 12 shows example brain maps obtained in an example use case based on the disclosed technology.

FIG. 13A shows examples of power per brain regions, as defined by the Harvard-Oxford 2 mm cortical atlas, obtained in an example use case based on the disclosed technology.

FIG. 13B shows examples of power per brain regions, as defined by the Harvard-Oxford 2 mm subcortical atlas, obtained in an example use case based on the disclosed technology.

FIG. 14 shows examples of statistical significance between SPECTRE power modes obtained in an example use case based on the disclosed embodiments.

FIG. 15 shows a flow diagram of an example method that can be carried out in accordance with an example embodiment.

FIG. 16 shows a flow diagram of another example method that can be carried out in accordance with an example embodiment.

### DETAILED DESCRIPTION

The human brain communicates internally through exceedingly complex spatial and temporal patterns of electrical signals. Although these signals can be measured using electrodes placed on the surface of the scalp (e.g., electroencephalography, or EEG), techniques to reconstruct the spatial and temporal patterns within the brain has been thwarted by the complexity of the inverse problem: What time-(or frequency-) dependent volumetric electrical signals throughout the brain are consistent with the signal measured on the two-dimensional surface of the scalp? At the heart of the problem has been the lack of a physical model for how electromagnetic (EM) waves propagate through the highly heterogeneous and anisotropic tissues of the brain. A more complete physical description of the dynamics of the propagation of electromagnetic (EM) waves through brain tissue reveals that the “volume conduction” limitation inherent for quasi-static EEG source models does not preclude developing statistically accurate volumetric maps of electric field distributions.

Disclosed herein are example embodiments that provide such a physical description: a universal framework of brain waves called weakly evanescent transverse cortical waves (WETCOW), that explains the broad range of observed but seemingly disparate brain spatiotemporal electrical phenomena from extracellular spiking to cortical wave loops. The disclosed embodiments, among other features and benefits, facilitate solutions of the EEG inverse problem through a reconstruction of brain electrical activity with high spatial and temporal resolution. In some implementations, the disclosed embodiments are capable of estimating time-dependent volumetric fields with a high level of statistical evidence from EEG data acquired on the surface of the skull. In some implementations of the disclosed embodiments, the WETCOW framework for brain wave propagation is employed to reconstruct and map fast-varying electric field distributions within the brain with high spatial and temporal resolution.

In an example embodiment, a method for solving the inverse EEG problem using WETCOW framework is disclosed. As previously discussed, the inverse EEG problem can be formulated as: what time-(or frequency-) dependent volumetric electrical signals throughout the brain are consistent with the signal measured on the two-dimensional surface of the scalp? In the description that follows, the method for solving the inverse EEG problem is demonstrated through an example use case involving a neuroimaging study of children with autism spectrum disorder (ASD), a complex neurodevelopmental disorder associated with early and pervasive deficits in selective attention. The study includes both EEG and functional MRI (fMRI) data, thereby allowing comparison with previous results obtained using the current standard for spatial localization (fMRI) of brain activity.

In the example embodiment described above, the disclosed method for solving the inverse EEG problem comprises at least some of the following steps. Given a standard EEG dataset from N electrodes and a high-resolution anatomical (HRA) MRI dataset with high contrast between gray matter (GM) and white matter (WM), the solution to the inverse EEG problem can be formulated as an approximation for the volumetric distribution of electrostatic potential inside the complex distribution of inhomogeneous and anisotropic tissues and complicated morphology of the MRI domain. A general form of this approximation is based on Maxwell's equations in an anisotropic and inhomogeneous medium expressed as charge continuity. This is used to derive the frequency dependent electrostatic field potential ¢ that is dependent upon the electrical properties of the tissue permittivity, permeability, and conductivity. These parameters can be estimated from the HRA data. In the Fourier (i.e., frequency) domain, the electrostatic potential satisfies the equation

$$( {\sum }^{ij} - I {ωεδ}^{ij} ) {\partial }_{i} {\partial }_{j} {ϕ}_{ω} = [ I ω ( {\partial }_{i} ε ) {δ}^{ij} - ( {\partial }_{i} {\sum }^{ij} ) ] {\partial }_{j} {ϕ}_{ω} + {ℱ}_{ω} , \qquad ( Equation 1 )$$

which is written in tensor form where a summation is assumed over repeated indices. This can be expressed in the form \hat{L}ϕ_{ω}=\hat{R}ϕ_{ω}+ [symbol] in terms of the operators \hat{L}≡∂ⁱ∂ᵢ, a frequency-dependent source term [symbol] and the operator

$$\hat{R} ≡ \frac{σ + I ωε}{{σ}^{2} + {ω}^{2} {ε}^{2}} [ I ω ( {\partial }_{i} ε ) {δ}^{ij} - ( {\partial }_{i} {\sum }^{ij} ) - ( {\sum }^{ij} - σ {δ}^{ij} ) {\partial }_{i} ] {\partial }_{j}$$

where Σ={Σ^{ij}} is a local tissue conductivity tensor, σ=TrΣ/3=Σᵢⁱ/3 is an isotropic local conductivity. Terms in square brackets show that the parts of \hat{R}ϕ_{ω} can be interpreted in terms of different tissue characteristics and may be important for understanding the origin of sources of the electro-/magnetostatic signal detected by the EEG sensors. The first term (ω(∂ⁱε)(∂ᵢϕ_{ω})) corresponds to areas with sudden change in permittivity, e.g. the WM/GM interface. The second term ((∂ᵢΣ^{ij})(∂ⱼϕ_{ω})) corresponds to regions where the conductivity gradient is the strongest, i.e. the GM/CSF (cerebral spinal fluid) boundary. Finally, the last term (Σ^{ij}∂ᵢ∂ⱼϕ_{ω}−σ∂ⁱ∂ᵢϕ_{ω}) includes areas with the strongest conductivity anisotropies, e.g. input from major WM tracts. The frequency and position dependent internal sources [symbol] can be used to incorporate various nonlinear processes including multiple frequency effects of the efficient synchronization/desynchronization by brain waves or effects of their critical dynamics.

The inverse problem can be solved by constructing an approximate solution for the potential ϕ across an entire brain volume iteratively as \hat{L}ϕ_{ω}^{(k)}=\hat{R}ϕ_{ω}^{(k-1)} and \tilde{ϕ}_{ω}^{(K)}=α_{K}Σₖ₌₀^{K}ϕ_{ω}^{(k)}, where a single iteration forward solution can be found using a Fourier-space pseudo-spectral approach. Data from MRI can be used to define the complex brain tissue morphology and constrain the tissue specific values of Σ and ε. This procedure of inverting the WETCOW brain wave model constrained by MRI-defined tissue properties is called SPatially resolved EEG Constrained with Tissue properties by Regularized Entropy (SPECTRE).

The disclosed method for solving the inverse EEG problem can be demonstrated through an example use case in which a neuroimaging study of children with ASD was performed. The ability of SPECTRE to directly detect, quantify, and visualize brain electric field networks suggests ASD as a natural use case for the method. ASD is diagnosed on the basis of social-communication impairments and the presence of restricted and repetitive behaviors, as well as early and persistent deficits in attention. While there is a large body of experimental neuroscience research focused on understanding the etiology of this disorder, the inability to directly reconstruct the spatially-localized real-time changes in the brain attention networks has significantly limited the ability to fully understand the neurofunctional mechanisms associated with the disorder.

As will be described, the disclosed method implemented in the example use case demonstrates the ability of SPECTRE to investigate the neurofunctional underpinnings of attentional differences observed in ASD by reanalyzing EEG data from a previously published study that used a rapid serial visual presentation (RSVP) task in a cohort of 21 children with ASD and 19 age—and nonverbal IQ-matched typically developing (TD) children. In the example use case, it is demonstrated that TD children exhibit a strong right-lateralized activation to behaviorally-relevant information in regions associated with the ventral attentional network; this pattern of activation was absent in ASD. Additionally, differences in cerebellar activation across groups are also present. These findings are in agreement with previously published fMRI data for an identical paradigm with the same cohort of participants and support the identification of spatially localized networks using the disclosed method. This comparison must be understood within the inherent limitations of fMRI, however, which is only indirectly related to the brain's electrical activity through a complicated coupling of local blood metabolism, flow, and oxygenation changes. Thus, the measured activations are not necessarily co-localized with the electrical activity and are insensitive to all but the very lowest frequencies. Moreover, these finding may not generalize to younger or lower function ASD as MRI in younger and lower-ability individuals on the autism spectrum is challenging due to the requirements necessary for image acquisition (e.g., remaining still for extended periods of time).

The data in this study were previously analyzed using standard EEG analysis methods to examine alpha-band oscillatory activity associated with attention and found reduced alpha desynchronization in children with ASD to the onset of behaviorally relevant targets compared to neutral targets. The study cohorts consisted of 19 children with ASD and 21 age—and non-verbal IQ-matched TD children. Participants completed a RSVP paradigm designed to investigate responses to behaviorally-relevant targets and contingent attention capture by task-irrelevant distractors, which either did or did not share a behaviorally relevant feature. Three simultaneous varying streams of numbers were presented and participants were tasked with identifying red numbers appearing in the central stream of colored numbers. Digits in the peripheral streams were gray except for occasional distractors colored the same as the target color (red) or a nontarget color (green). For the purposes of the study, only the target-present neutral trials were analyzed (i.e., red number in the center flanked by gray distractors).

Continuous EEG was recorded using a BioSemi ActiveTwo system (BioSemi B.V., Amster-dam, the Netherlands) with 68 Ag/AgCl active electrodes. Sixty-four electrodes were mounted in an elastic cap according to locations in the modified International 10-20 system. The remaining electrodes were placed below the right eye, on the outer canthus of the left eye (to monitor blinks and saccades), and over the left and right mastoids (reference). EEG data were recorded at a sampling rate of 256 Hz, and direct current offsets were kept below 25 mV at all channels. Data were pre-processed using EEGLAB. Data were re-referenced to the average of the left and right mastoid electrodes, and high-pass filtered at 0.5 Hz, and decomposed using extended Info-Max independent component analysis. Components reflecting highly stereotyped oculomotor artifacts (e.g., blinks, saccades) were removed using a standard independent components analysis artifact removal procedure. Three-second epochs (−1000 to 2000 ms around trial onset) were extracted from the artifact-cleaned continuous EEG data for correct trials for the target present/neutral distractor (P-N) condition. Finally, epochs were rejected if they exceeded a threshold of 150 mV or a sample-to-sample threshold of 100 mV. 3-second epochs were further divided into pre-(−1000 to 0 ms) and post-target (0-2000 ms) time windows in order to examine event-related changes in alpha-power sources/connectivity related to the onset of the target (i.e. red number in the center stream). This technique allows for determination of changes in activation/connectivity related to processing of behaviorally-relevant information. All images were registered using SYMREG to the standard MNI2 mm brain, which was used as the input for brain morphology and tissue property determination.

The k′th EFD mode of the coupled parameters α={α₁, . . . , αₘ} is denoted ψ_{α}^{(k)} (x,t) where k=1, . . . , n for some user defined n. The STIT generated from the modes are denoted F_{α}^{(k)} (x,t). For each STIT connectivity, eigenmodes (CEM) C_{α}^{(k,l)} (x,t) are calculated from l=1, . . . , r for a user defined r. For each subject n=10 power modes ψ_{α}^{(k)} (x,t) were reconstructed and summed over all modes \tilde{ψ}=Σₖⁿψ_{α}^{(k)} (x,t). Space-time information trajectories (STITs) F_{α}^{(k)} (x,t), k=1, . . . , n were also calculated for each EFD mode. For each STIT, r=5 connectivity eigenmodes (CEM) were calculated, then summed over all modes. Therefore, for the summed (over k) power modes, summed over the l connectivity eigenmodes, a single CEM volume denoted \tilde{C}=ΣₖⁿΣₗ^{r}C_{α}^{(k,l)} (x,t) was calculated for each subject.

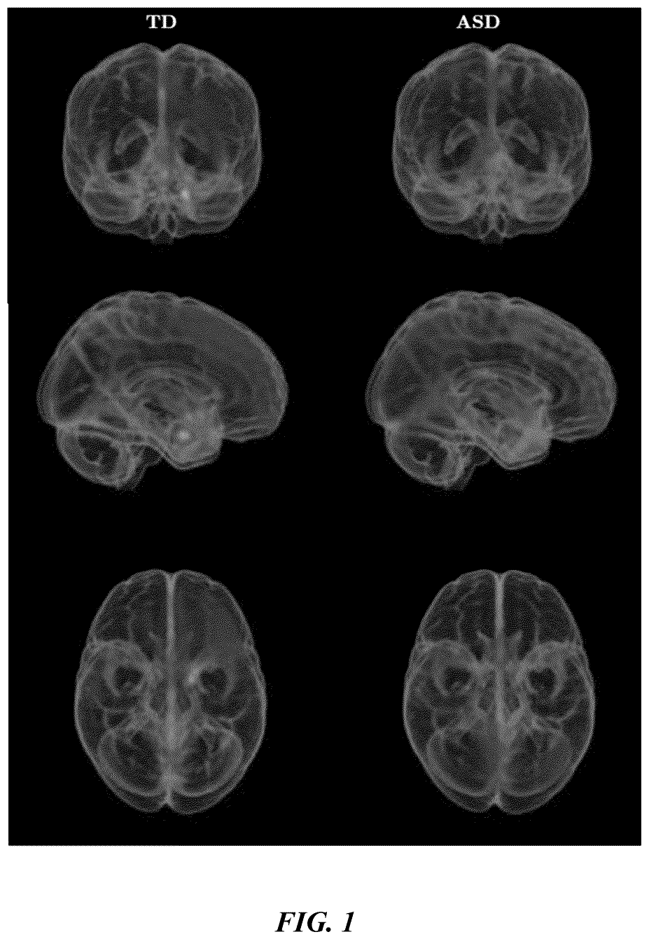

FIG. 1 shows example differences in SPECTRE power modes in the alpha band pre- and post for the Present-Neutral condition averaged over all participants in each cohort of the example study. The absolute value is shown (top rows) to highlight the spatial variations, and the mean difference (bottom rows) highlight the activated (red) and deactivated (blue) regions. Pre-versus post-target difference maps show greater target-related activation (red) in TD children; children with ASD displayed diffuse pattern of deactivation (greater pre-compared to post-target alpha power).

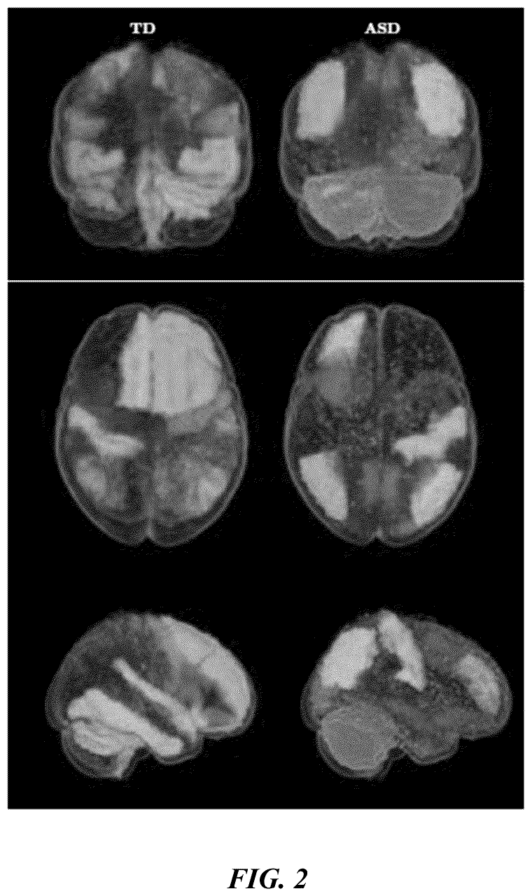

FIG. 2 shows an example of a power difference atlas used in the example study. The power difference atlas was constructed using the Haskins pediatric atlas in MNI space. For each cohort, the histogram of positive and negative power differences (FIG. 1) in each voxel were calculated and then each brain region was assigned to the classification (positive or negative) based on whichever had the greater probability. Power difference atlas results show that TD children exhibit target-related activation of key nodes in the right-lateralized ventral attentional network, including temporal-parietal and inferior frontal cortex. Further, also similar to our previous fMRI results, cerebellar activation was also present. Children with ASD showed relatively limited target-related activation, primarily restricted to medial prefrontal cortex.

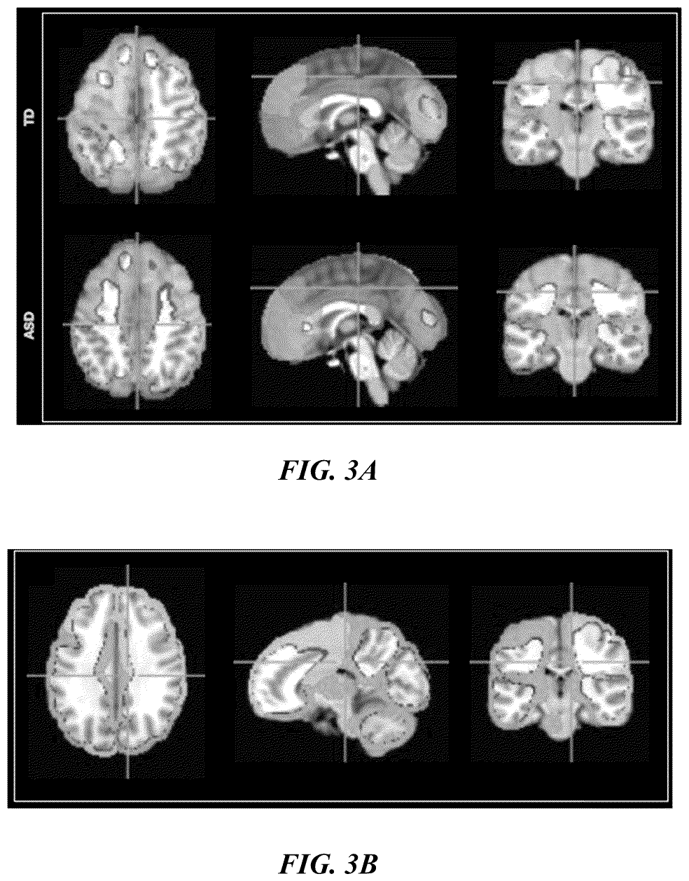

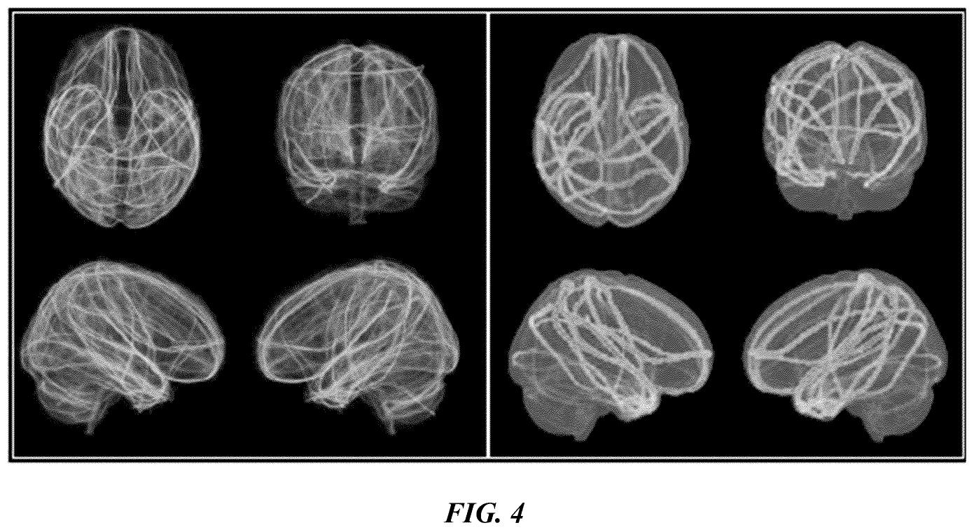

FIG. 3A shows example images, in a slice representation, of statistical significance between the pre and post conditions within the two cohorts of the example study. A standard t-test was implemented using the program 3dttest++ in AFNI. The threshold for significance is p≤10⁻⁸ and thus highly significant. For direct comparison between TD and ASD cohorts, the statistical significance via t-test was computed between the pre and post condition differences in the two cohorts, as shown in in a slice representation in FIG. 3B. The threshold for significance is p≤10⁻⁸ and thus highly significant.

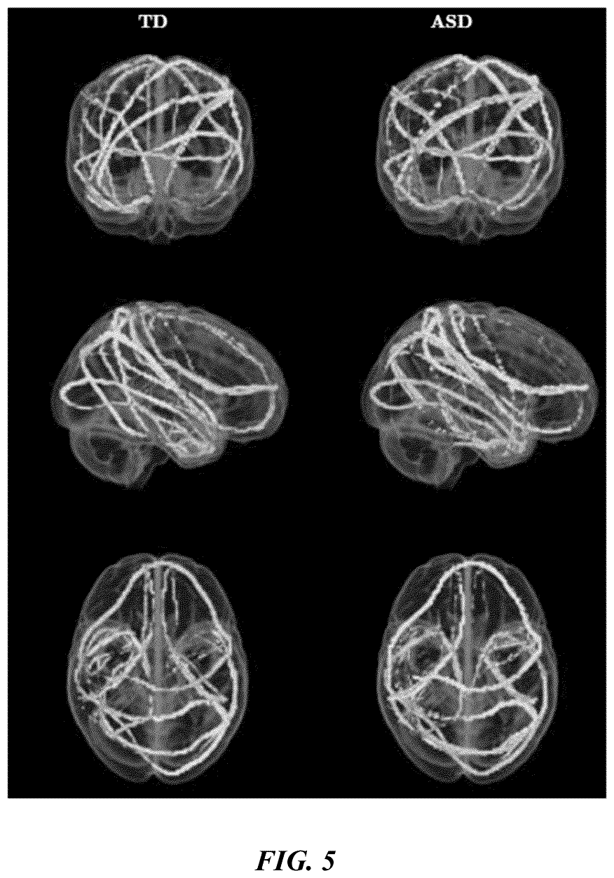

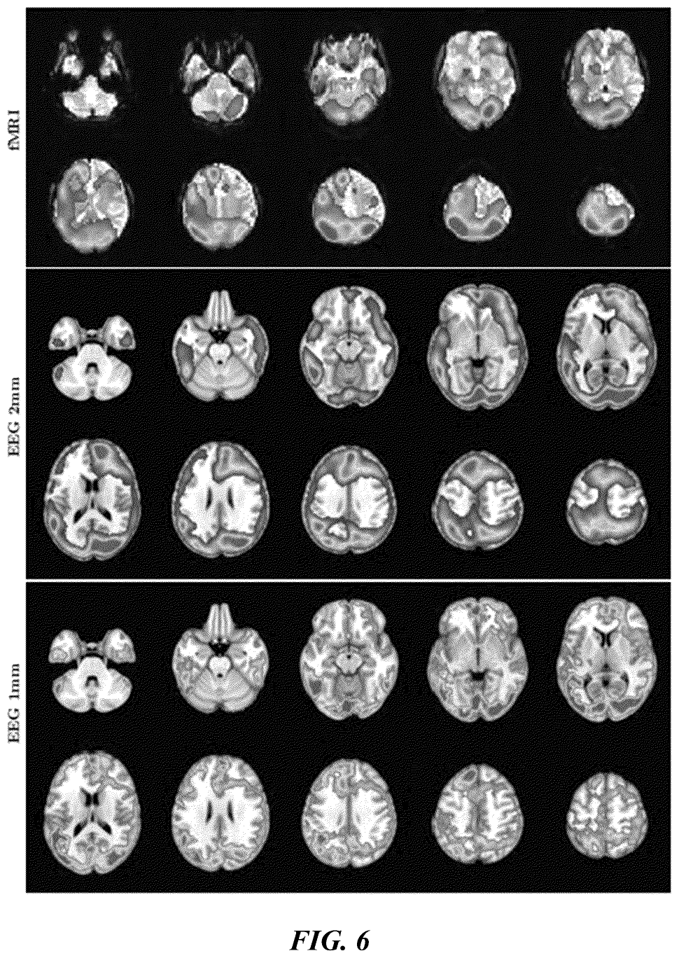

FIG. 4 shows an example of the STITs F_{α}^{(k)} (x,t) and the associated CEM \tilde{C} for a single subject as an illustration of the integrity of the single subject results SPECTRE produces. The calculations for all subjects are remarkably similar, a testament to the consistency of the computation across subjects, and important feature critical for detecting subtle network differences, which are shown in the example STIT differences pre-post shown in FIG. 5.

The disclosed SPECTRE methodology thus provides the ability to investigate the spatially and temporally resolved electrical activity in children with ASD and compare them to a cohort of TD children, as was demonstrated in the example use case. Previous analysis of this data using a standard channel-based EEG analytic approach demonstrated reduced alpha desynchronization in children with ASD to the onset of behaviorally relevant targets compared to neutral targets. These observations had essentially no spatial information other than they appear dominant in the posterior electrodes. From this it was concluded that reduced neural activation to target stimuli may be associated with atypical activation of ventral attention network. Using SPECTRE, the spatial extent of this range of frequency-dependent signals is uncovered which confirms the hypothesized ventral attention network activation and demonstrates consistency with prior fMRI results. The analysis revealed significant differences in the brain networks in the alpha range 8-12 Hz, consistent with previous spatially resolved fMRI experiments but with far great temporal (or frequency) resolution. In the example use case, focus on this alpha range was for comparison, but the analysis is easily carried out in any user-defined frequency ranges. Implementations of the disclosed SPECTRE methodology are applicable to any EEG study and thus hold promise for a wide range of ongoing studies including studies of ASD, Alzheimer's Disease, Epilepsy and a multitude of other scientific and clinical neuroscience applications.

One advantage of SPECTRE is that SPECTRE is flexible in its ability to incorporate relevant prior information from MRI data. HRA data is useful for tissue segmentation and assignment of mean values for permittivity and conductivity. Diffusion MRI (dMRI) data further allows construction of estimates of the conductivity tensor anisotropy. In the example neuroimaging study, only HRA data were used. One important practical point is that very often HRA data are not available for participants in EEG studies. In this case it is sufficient to use HRA data from a standard atlas, and spatially register the EEG data to the atlas. In the example neuroimaging study, the standard 2 mm resolution T1-weighted anatomical MRI Montreal Neurological Institute (MNI) atlas was used, to which the EEG data were aligned using a non-linear registration algorithm (SYMREG) described elsewhere.

To emphasize some of the benefits of the disclosed method, the method may be contrasted with current so-called “source localization” methods that involve numerous stringent (and not physically motivated) assumptions about brain electrical activity such as a fixed set of static dipole sources, an idealized geometric model of the head reduced to a few (typically 3) shells, that spatially close points are more likely synchronized, and the smoothness of the solution. These methods all implicitly assume the “quasi-static” approximation to the EM field equation which entails ignoring the time dependent terms in Maxwell's equations, which are dependent on tissue conductivity properties which are themselves frequency dependent. The resulting solutions are therefore static, have no frequency dependence, and are insensitive to the detailed spatially variable electrical properties of the tissues. However, by solving the actual physical problem of the complete Maxwell's equations in an inhomogeneous and anisotropic medium using the disclosed method, it is specifically these dependencies that give rise to the previously undiscovered WETCOW waves that propagate preferentially along the gradients of local tissue inhomogeneity and anisotropy. The method reveals the previously undiscovered fact that waves propagate preferentially perpendicular to neuronal pathways. Thus, disclosed embodiments based on the WETCOW framework can provide a comprehensive framework for characterizing the propagation of EM fields through the complex tissue microstructure and larger scale morphology (e.g., cortical folding), and provide the dynamic solution to the electric potential field necessary to solve the EEG inverse problem. Additionally, it has long been recognized that the two primary functional neuroimaging modalities, fMRI and EEG, offer strengths and weaknesses: spatial localization in fMRI with poor temporal resolution, high temporal resolution in EEG with the inability to accurately spatially localize signals. The limitations in fMRI are fundamental, as the signal changes are directly related to blood oxygenation changes and local flow dynamics and metabolism which occur on a very slow time scale relative to the actual neural dynamics. The ability to spatially localize EEG using implementations of the disclosed embodiments would not only allow the direct mapping across the entire frequency range of brain electrical activity, but also more refined comparisons with fMRI.

The disclosed embodiments, among other features and benefits, provide a pseudo-spectral computational approach to solving the inverse EEG problem which has some important advantages over the finite/boundary element approaches typically used for electrostatic modeling of brain activity. The pseudo-spectral computational approach does not use surface meshes and so does not require limiting the location of sources to a small number of surfaces with fixed number of static dipole sources constrained to the surfaces. Additionally, the distribution of both electrostatic and geometric properties of the media (e.g., conductivity, permittivity, anisotropy, inhomogeneity-derived from the MRI data) are incorporated at every location throughout the volume. Thus, the pseudo-spectral computational approach is able to find a time dependent spatial distribution of the electrostatic potential at every space-time location of a multidimensional volume as a superposition of source inputs from every voxel of the same volume. These traits allow it to model wave-like signal propagation inside the volume and to detect and characterize significantly more complex dynamical behavior of the sources of the electrostatic activity recorded at the sensor locations in comparison to traditional methods.

To facilitate understanding of why SPECTRE is capable of reconstructing EM activity through the entire brain, including deep within subcortical structures, a simple idealized example is presently described. Consider two point current sources of different frequencies, one in the cortical layer close to the scalp, the second deep within the subcortical structures of the brain. A single sensor is placed on the scalp collinear with the two sources. Standard source localization methods will not see the deep source, since there is no frequency dependence, and the signal falloff is simply a function of the distance from the sensor. Therefore, the close source completely dominates the signal model. All tomographic imaging methods (e.g., MRI, CT, etc.) depend strongly on both the spatial and temporal sampling of the measured physical system. This effective invisibility of currents in the standard quasi-static model essentially precludes the solution of the true inverse EEG problem and necessitates the artificial construction of assumed dipole distribution on pre-chosen artificial internal structures. In contrast, in SPECTRE the sources are not dipoles, but frequency sources that extend throughout the entire brain volume subject to the boundary conditions imposed by both the tissues geometry and its spatially and frequency dependent properties. The surface electrodes are assumed to be sensing EM waves across a broad frequency spectrum limited only by the sensors and emanating from the entire brain. Used in conjunction with an HRA MRI data that provides the spatial distribution of the frequency-dependent tissue electrical properties that constrain the possible solution, SPECTRE can invert the wave equations to provide an estimate of the spatiotemporal distribution of the electric field potential. The reconstructed volumetric time series of the estimated electrostatic potential field can be thought of as similar to the EM equivalent of an fMRI dataset, containing a multitude of correlated spatiotemporal patterns or “modes” of the system. The problem then becomes one of detecting the multiple modes in complex non-linear systems. This problem has been addressed using entropy field decomposition (EFD) which will be discussed in the description that follows.

In another example embodiment, a method for direct imaging of the electric field networks in the human brain from EEG data is disclosed. Among other features and benefits, the method can provide much higher temporal and spatial resolution that fMRI, without the concomitant distortion. In the description that follows, the method is validated using simultaneous EEG/fMRI data in healthy subjects, intracranial EEG data in epilepsy patients, and in a direct comparison with standard EEG analysis in a well-established attention paradigm. As will be described in further detail, the method is demonstrated in an example use case involving a very large cohort of subjects performing a standard gambling task designed to activate the brain's ‘reward circuit’. The disclosed technique uses the output from standard EEG systems and thus has potential for immediate benefit to a broad range of important scientific and clinical questions concerning brain electrical activity, while also providing an inexpensive and portable alternative to fMRI.

### 1. Example Methods

Despite the highly dynamical nature of the electrical activity that occurs within the very inhomogeneous and anisotropic composition of brain tissue, current EEG data analysis methods are still based on the assumption that the average tissue bioelectric properties (e.g., the average permittivity ϵ̅₀̅ and conductivity σ̅ are sufficient to describe the electric fields/in the brain. This leads to the approximation |ϵ̅₀̅∂E/∂t|<<|σ̅E| of which in turn leads to the assumption that the time dependence ∂E/∂t can be ignored in the “typical” frequency range of brain signal. This is the ubiquitous so-called “quasi-static” approximation. In reality, it is precisely the anisotropic and inhomogeneous nature of brain tissue that must be taken into account in order to develop an accurate physical model of brain electromagnetic behavior, as previously described in this patent document in connection to WETCOW framework. A consequence of WETCOW framework is the existence of electric field waves generated as a consequence of the complex tissue boundaries (e.g., surface waves) that permeate throughout the brain and are in precisely the frequency range of observed brain electrical activity. WETCOW framework explains the broad range of observed but seemingly disparate brain spatiotemporal electrical phenomena from extracellular spiking to cortical wave loops, all of which are predicated on the time dependence of the electric fields within the complex architecture of anisotropic and inhomogeneous tissue within the brain. As discussed above, WETCOW framework is necessary to provide a solution to the EEG inverse problem which, as will be discussed in the description that follows, produces a reconstruction of brain electrical activity with high temporal resolution and spatial resolution that is comparable (or even exceeding that of) functional MRI.

In an example embodiment, a method for direct imaging of the electric field networks in the human brain from EEG data reconstructs the electric field potential throughout the entire brain using the WETCOW wave model constrained by MRI-defined tissue properties. This approach is based on SPECTRE, which has been described above, and solves a dynamic time-dependent construction of Maxwell's wave equations of electromagnetism in an inhomogeneous and anisotropic medium. Thus, the approach is distinctly different than standard so-called “source localization” methods.

One problem of spatially localizing an EEG signal involves estimating the most probable distribution of electric field amplitudes given an array of sensors. This is essentially a problem of correctly modeling the physics of how electromagnetic waves propagate through the complex environment of the convoluted brain tissue morphology and the anisotropic and inhomogeneous nature of brain tissue. The current state-of-the-art approach to this problem is “source-localization” (e.g., low resolution electromagnetic tomography or LORETA algorithm with its many variations, also called ‘EEG source imaging’) which involves using a pre-defined brain atlas, arbitrarily placing dipole sources on the surface, and calculating the contribution from these sources. The current source localization methods are based on a static model for the electric field caused by a fixed set of pre-defined dipole sources and are thus inherently limited because, in reality, the brain's electrical field variations are time dependent and generated by an essentially continuous distribution of sources throughout the entire brain. In contrast to “source-localization” techniques, WETCOW framework describes how highly coherent localized electric field phenomena, such as cortical wave loops and synchronized spiking, are produced by the complex non-linear interactions of waves across multiple spatial and temporal scales. In some disclosed methods based on SPECTRE, MNI volumetric grid with 2 mm (902629 voxels), 1 mm (7221032 voxels), or 0.73 mm (11393280 voxels) are used. All voxels in the models are considered sources of electromagnetic activity consistent with the local intravoxel tissue characteristics rather than an assumed dipolar form.

### 2. Example Validation

Validation of any neuroimaging method is problematic because it is not possible to directly measure brain activity at every location in the brain. Nevertheless, three methods are candidates for assessment of SPECTRE's validity.

The first is comparison with fMRI, the current method of choice for whole brain spatial localization of brain activity. However, association of fMRI with a “standard” for EEG is problematic because it is not measuring electrical activity, but the magnetization changes in hemoglobin as blood becomes deoxygenated during brain activity. The timescale and location of these changes can be vastly different than those produced by EEG signals. Nevertheless, its capability of spatially localizing activated brain regions merits a comparison. The most direct comparison is between fMRI and EEG data collected simultaneously, which guarantees that the brain activity measured is identical in both experiments. Such “simultaneous fMRI/EEG” experiments are not particularly common as collecting EEG data within an MRI scanner during imaging is notoriously difficult, and the MR imaging procedure significantly distorts the EEG signal.

A more direct method for validating the ability of SPECTRE to reconstruct localized electrical activity can be constructed from intra-cranial EEG (iEEG) recordings collected during epilepsy studies. Such measurements consist of specially designed EEG sensors distributed linearly along a probe that is inserted deep within a brain that has been exposed by surgical removal of a portion of the skull. By selecting only these electrodes near the brain surface from the full array of electrodes, an artificial surface distribution of electrodes to mimic a standard non-invasive EEG experiment can be synthesized (albeit with a limited coverage of the brain).

Lastly, a comparison with current “source localization” methods can be performed. This comparison turns out to be the most problematic as these methods all employ a very different, and quite limited, physical model for the EEG signal, and suffer from computational limitations as well.

2.1 Example Validation with Simultaneous fMRI/EEG Visual Task

It is notoriously difficult to get high quality EEG data in simultaneous fMRI/EEG studies as the presence of the rapidly varying magnetic fields present in an fMRI acquisition distort the EEG signal. However, one recent open-source simultaneous fMRI/EEG study of a well-controlled visual task (the periodic flashing checkerboard) on multiple subjects provides important data to address this question.

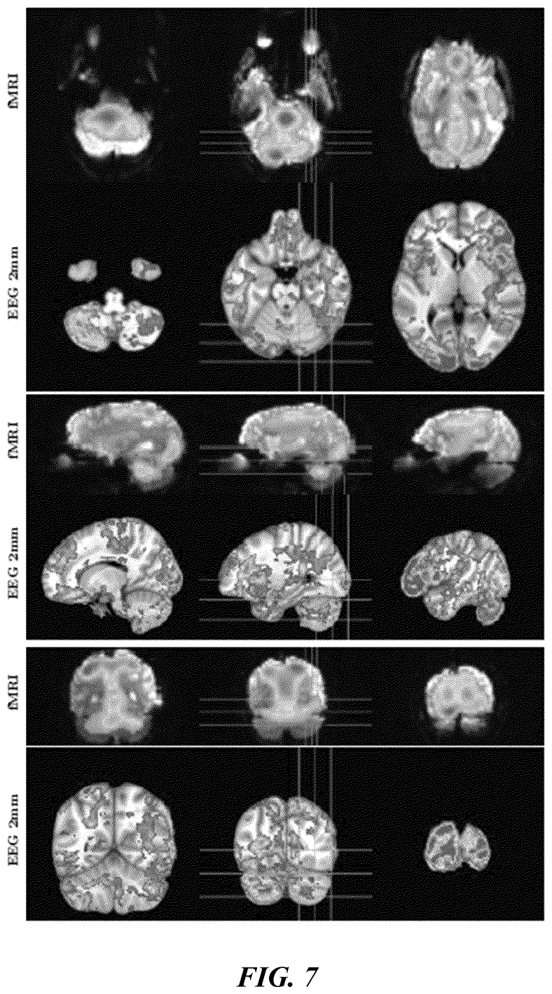

The ability of the disclosed method, based on SPECTRE, to faithfully reconstruct the spatial distribution similar to fMRI is shown in FIG. 6. Importantly, this comparison was performed on data from a single subject, since brain activity patterns can vary significantly between individual and averaging over multiple subjects obscures specific spatial variations important for validation. In the top rows of FIG. 6 are shown the fMRI EFD mode that automatically detects the activation in the primary visual cortex. In the middle row are shown the SPECTRE modes reconstructed using the 2 mm MNI anatomical atlas, chosen because it was closest in resolution (2 mm³) to the fMRI data (˜3 mm³). Very close correspondence between the spatial patterns is evident from FIG. 6.

The bottom rows in FIG. 6 demonstrate an additional aspect of SPECTRE-its ability to reconstruct activation at spatial resolution significantly higher than fMRI. This is a consequence of the SPECTRE reconstruction being based on the solution of the propagation of electromagnetic wave through specific tissue morphologies and bioelectric properties, provided by arbitrary resolution anatomical MRI data. The finer the resolution of the MRI scans, the more details can be available for the reconstruction. This is of course dependent upon the number and distribution of the EEG sensors, but certainly holds for the standard array configurations used disclosed embodiments.

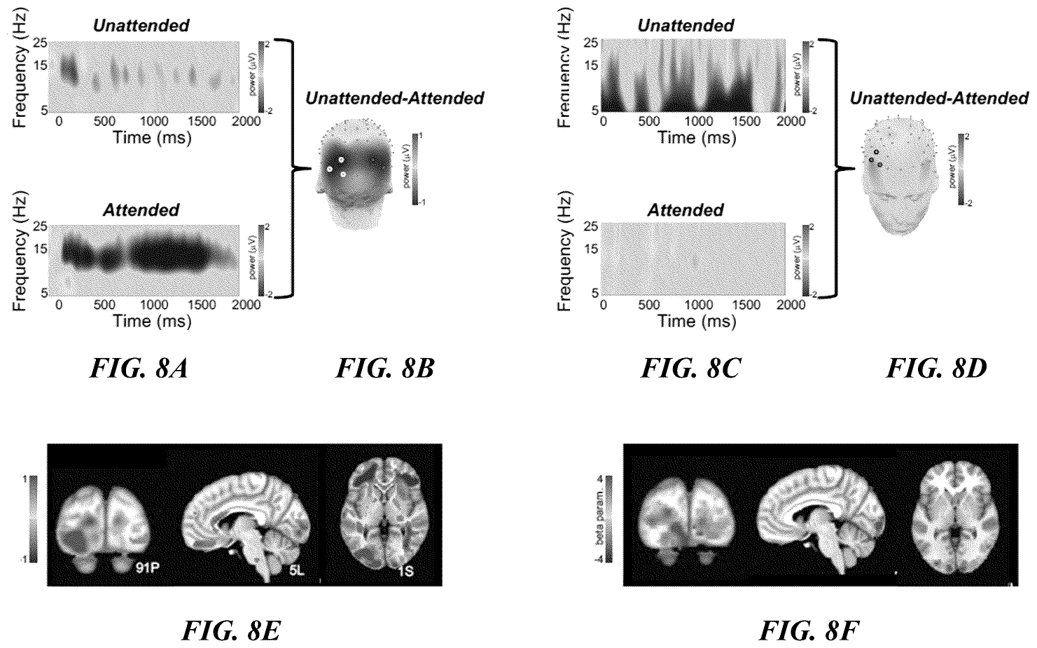

Although it is a commonly believed notion that EEG and fMRI are complementary because EEG has excellent temporal resolution but poor spatial resolution, while fMRI has poor temporal resolution but good spatial resolution, in fact SPECTRE EEG reconstructions can achieve much higher intrinsic temporal and spatial resolution. Moreover, because there are no spatial distortions in SPECTRE, this mitigates one of the aspects of fMRI that most confounds spatial localization through signal loss and non-linear geometric distortions. This is shown in FIG. 7 which illustrates an example comparison of EFD reconstructed fMRI activity (top) with SPECTRE reconstructed EEG at 2 mm (bottom) from a single subject.

It should be noted that the “simple” periodic flickering checkerboard stimulus not only activates the primary visual cortex but activates other visual and supplementary fields as well, as is evident from the activity patterns in FIG. 6. A simple stimulus does not imply a simple activation pattern. The activation mode reconstructions for both the fMRI and SPECTRE data are based on the EFD which detects complex non-linear interacting spatial-temporal modes of activity. Thus, although the task is a “simple” visual stimulation, our analysis is not expected to simply detect activity in only the visual cortex, as would be produced by a more standard regression approach, but in a more complex set of brain networks. Indeed, multiple EFD modes are produced, though we have only shown the one incorporating the primary visual cortex in FIG. 7. It has been demonstrated that EFD analysis is more sensitive than simple regression techniques to the complex brain activation patterns predicted by neuroscience, and less sensitive to erroneous identification of noise or non-independent modes than the independent component analysis (ICA). Indeed, one observation from both the fMRI and EEG data used in this study is the appearance of prefrontal cortex (PFC) activations associated with visual stimulation, which has been suggestive of conscious visual perception.

2.2 Example Validation with Simultaneous fMRI/EEG Attention Paradigm

Simultaneous EEG/fMRI were collected from subjects within a standard clinical 3T MRI scanner (see Section 2.2.a for details). The stimuli and paradigm have been described in detail elsewhere. Briefly, bimodal stimuli consisting of short (˜1 s) streams of simple tones (600 and 1000 Hz) alternating at 10 Hz were delivered concurrently with phase-reversing (6 Hz) checkerboard patterns presented at fixation. Participants were instructed to selectively attend to either the visual or auditory aspect of the bimodal stimulus and respond when the stream of stimuli in the attended modality ends.

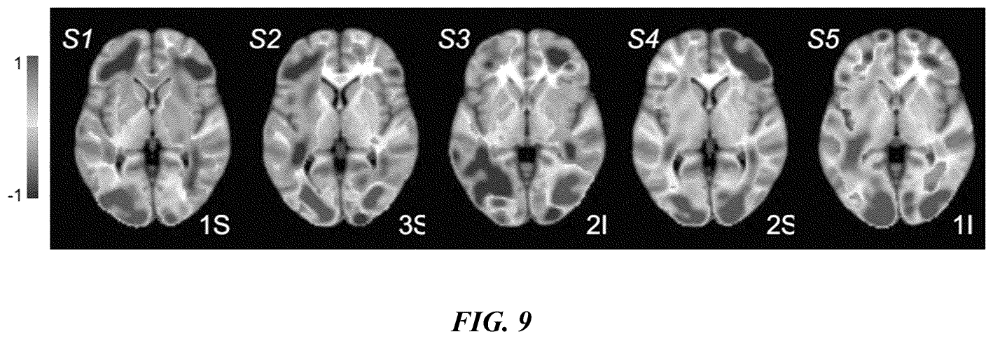

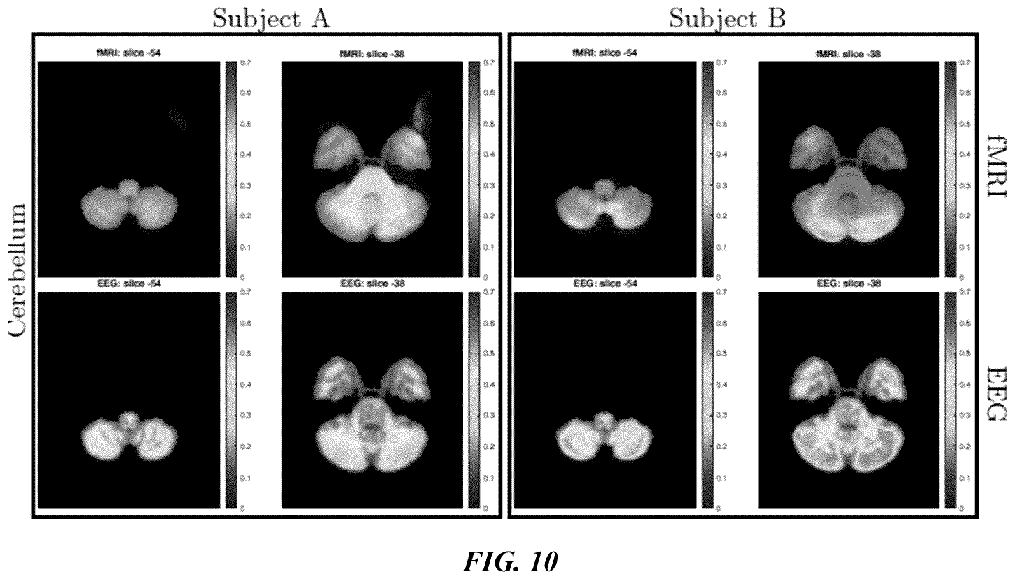

SPECTRE processing was performed in the alpha band. The appearance of visual stimuli elicited a reduction of ongoing alpha (7-14 Hz) activity (“event-related desynchronization”, ERD) over occipital cortex, believed to occur when cortical regions are brought “on-line” for information processing. As in previous studies, attended visual stimuli elicited increased (more negative) amplitude of the alpha ERD compared to unattended stimuli (FIGS. 8A and 8B). In contrast, unattended, compared to attended, visual stimuli elicited a greater reduction in ongoing spectral activity within the 5-15 Hz frequency range over bilateral middle frontal cortex (FIGS. 8C and 8D). The neural sources of these attention-related modulations of oscillatory activity were estimated across the 8-12 Hz frequency band which encompassed both the occipital and frontal activities (FIG. 8E). Their anatomical localization was remarkably consistent across several individuals (FIG. 9).

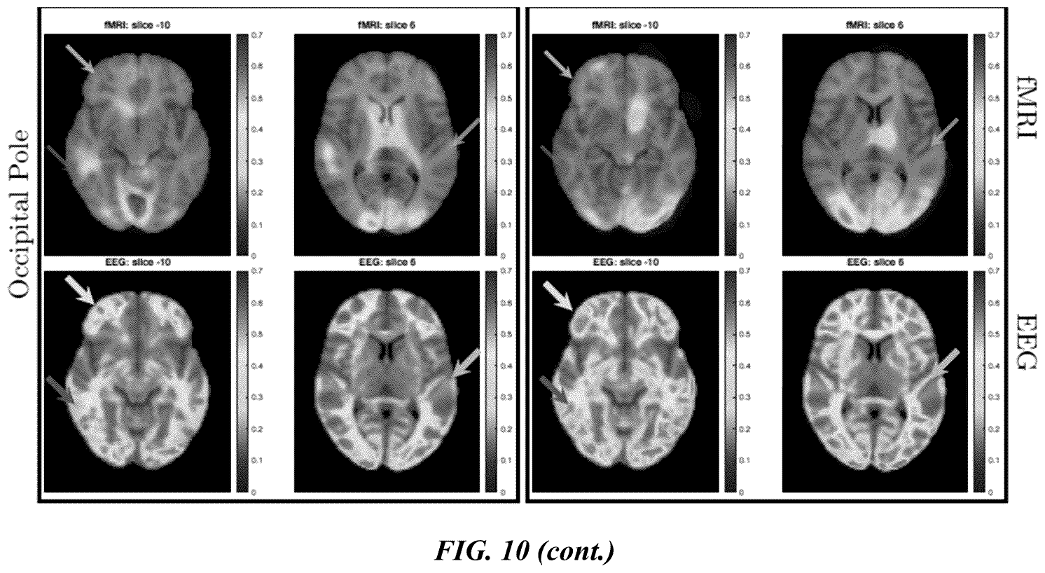

A direct comparison of the activation maps derived from both fMRI and EEG using SPECTRE for single study within two subjects (i.e. without any average over studies or subjects) is shown in FIG. 10. The comparison is made by choosing specific regions of interest defined in the MNI atlas (occipital cortex and cerebellum) and correlating the activation maps derived from EFD for fMRI and SPECTRE from EEG. Comparison of the similarity of activated regions in individual subjects is generally a non-trivial problem. This is particularly true in the current case where the spatial distortions in fMRI (and lack of them in SPECTRE) make measures such as mean-squared error difficult to interpret. Therefore computation of the correlation coefficient over a predefined atlas ROI is a reasonable conservative measure. The high correlation coefficients between the maps in FIG. 10 are therefore indicative of the consistency between the fMRI and SPECTRE results in the ROI. Note that this does not imply similarity over the entire region shown. Indeed, the SPECTRE results show enhanced sensitivity to activation in regions not seen in the fMRI.

### 2.2.a Example Attention Paradigm Data

Simultaneous EEG/fMRI acquisition: Functional and structural MRI images were acquired on a Siemens 3T TIM-Trio scanner (NKI Center for Biomedical Imaging and Neuromodulation) equipped with a 32-channel phased array head coil. Structural T1 and T2 scans were collected using standard sequences. Whole-brain BOLD data was acquired with a gradient-echo EPI sequence (TR=2000 ms; TE=30 ms; flip angle=80°). EEG data were acquired concurrently with fMRI using an MR compatible EEG amplifier (Brain Vision MR series, Brain Products, Munich, Germany) and a 64-channel MR-compatible ring electrode cap with 10-20 International System electrode placement cap. EEG data was sampled at a rate of 5 kHz EEG data were acquired at a rate of 5 kHz using Brain Vision Recorder software. Electrocardiographic data were captured from electrodes on the backs of subjects. The reference electrode was positioned between Fz and Cz. Scanner and heartbeat artifacts were removed offline from the EEG signal using an average template subtraction procedure and the data was resampled to 250 Hz.

Traditional EEG analysis: The single-trial EEG signal from each electrode was convolved with a 3-cycle Morlet wavelet computed over a 3 second window centered at the onset of each stimulus and averaged separately for each stimulus type. The averaged spectral amplitude at each time point was then baseline corrected by subtracting the mean spectral amplitude over the −200 to −50 pre-stimulus interval.

### 2.3 Example Validation Through Intra-Cranial EEG Recordings

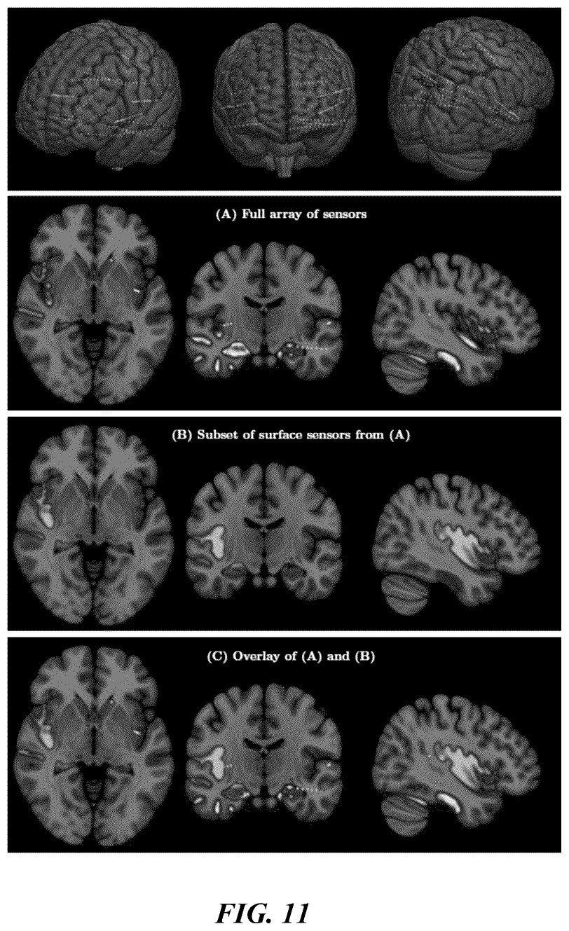

While comparison with fMRI can validate the correct detection of activated brain regions and networks, as shown in the previous section, it cannot inform the question of correct detection of electrical signals, since fMRI is based on a completely different contrast mechanism related to blood oxygenation. A direct validation of SPECTRE's ability to faithfully reconstruct deep electromagnetic activity is, to our knowledge, only achievable with one type of data: IEEG recordings such as those used in medically refractory epilepsy patients for seizure onset localization where the electrodes are known to be adjacent to the site of electrical activity. An HEEG recording of a seizure localized in the left mesial temporal region acquired at Northwell Health, NY was analyzed for the purposes of validating SPECTRE. All implanted electrodes are shown in FIG. 11 (top row) with each yellow dot depicting one recording contact. Comparing the SPECTRE reconstruction using all of the sensor data with one using only a subset of the data comprised of only the sensors on the surface of the brain (red dots in FIG. 11 (top row)) allows the quantitative assessment of how closely the results from a set of surface electrodes correspond to those produced by intra-cranial measurements recording signal very close to the sources. The results are shown for the alpha frequency band in FIG. 11 and reveal a very close correspondence between the SPECTRE mode reconstruction.

### 3. Example Investigation of the ‘Reward Circuit’

Having validated the SPECTRE method directly with simultaneous fMRI/EEG, iEEG, and an attention paradigm, an investigation was performed regarding the ability of SPECTRE to faithfully reconstruct the well-known neural “reward circuit” that is one of the most important in understanding human cognition, emotion, and behavior and is of great clinical significance in the understanding of addiction, mood disorders, and a variety of other conditions.

It is demonstrated that SPECTRE using standard EEG data can accurately map human reward pathways akin to results previously only seen via fMRI. Indeed, fMRI results have highlighted a reward system within the brain that includes midbrain dopamine producing regions (the substansia nigra pars compacta, the ventral tegmental area), the ventral striatum, and multiple regions within the human prefrontal cortex. Other research using fMRI and source localization of EEG data suggests that the anterior cingulate cortex also plays a key role in reward processing. In a unifying theory, it has been proposed that all the aforementioned regions work together as a neural system for the optimization of reward driven behavioral change (i.e., reinforcement leaning)

This is of particular clinical significance because addictive behaviors have long been known to be subserved by specific brain regions operating in concert as the reward circuit. The reward circuit is involved in processing rewarding stimuli of any sort and in drug addiction, substances of abuse (e.g., amphetamine) increase dopamine release in a protracted and less regulated manner as compared to typical stimuli, resulting in synaptic plasticity, and altered functioning of this circuit over time.

For the analysis described herein, a large gambling task dataset was used that includes 500 participants. The details of the dataset and an extensive analysis using standard EEG analysis methodologies have been presented elsewhere. In the study, participants completed a simple gambling task. On each trial, they saw a black fixation cross for 500 ms, followed by two colored squares for 500 ms, and then, the fixation cross turned gray (go cue) and participants were to select one of the two squares (square locations—left, right—were randomized on each trial) within a 2,000 ms time limit. They were then presented with a black fixation cross for 300 to 500 ms, and then, simple feedback as to their performance (“WIN” for gain, “LOSE” for loss) for 1,000 ms in black font. If the participants responded before the go cue they were instead delivered “TOO FAST” feedback and if they did not respond before the 2,000 ms time limit, it would be considered a loss. The goal of the participants was to accumulate wins by determining which of the two squares would more often lead to gains (60% vs. 10%). In this task, participants accumulated wins; however, were not paid money. They would see the same pair of colors for one block of 20 trials. They conducted six blocks of unique color pairs.

Participants included five hundred undergraduate students recruited via the University of Victoria psychology participant pool. The data was collected until 500 participants became available that were not characterized by one of the following a priori criteria: trial count after artifact rejection were less than 15 per condition, total artifact rejection exceeded 40% of trials rejected, FCz (electrode of interest) specific artifact rejection exceeded 40% of trials rejected, or independent component analysis based blink correction failed. These criteria were extremely strict to ensure clean data in the analyses, and as such a total of 637 participants were analyzed before reaching the goal of 500 clean participants. All participants had normal or corrected-to-normal vision.

EEG data were recorded from either a 64 or 32 electrode (Ag/AgCl) EEG system using Brain Vision Recorder. During recording, electrodes were referenced to a common ground, impedances were kept below 20 kΩ on average, data were sampled at 500 Hz, and an antialiasing low-pass filter of 245 Hz was applied via an ActiCHamp amplifier. Stimuli and EEG markers were temporally synced using a DataPixx stimulus synchronization unit.

Data were re-referenced to an average mastoid reference and filtered using a 0.1 to 30 Hz passband (Butterworth, order 4) and a 60 Hz notch filter. Correction for eye blinks was per-formed using EEGLAB's independent component analysis (ICA). Components reflective of blinks were manually identified and removed via topographic maps and component loadings, and data were reconstructed. Data were then segmented from −500 to 1, 500 ms relative to feedback stimulus onset, baseline corrected using a −200 to 0 ms window, and run through artifact rejection with 10 μV/ms gradient and 100 μV maximum-minimum criteria. Data were pre-processed to identify noisy or damaged electrodes using artifact rejection trial removal rates for each electrode.

The 1 second of recorded sequence for each “WIN” or “LOSE” event were extracted from recordings for each participants (with 22 ms of pre-event sample and 488 ms of post-event sample) and combined together to form separate winning and losing datasets. Each of those datasets were processed using SPECTRE to construct the approximate inverse solution for the potential φ across an entire 2 mm MNI brain volume.

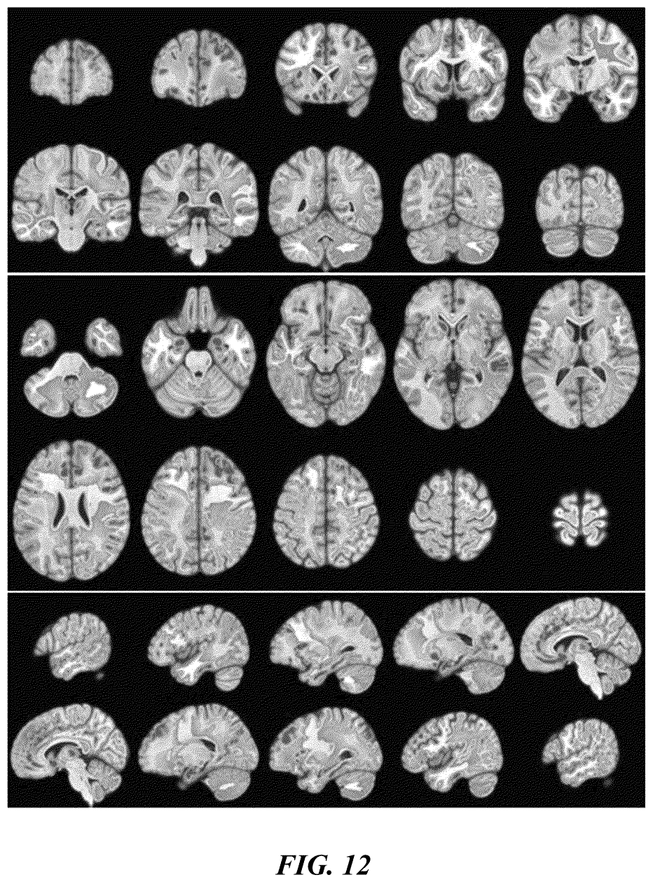

For each subject trial n=10 power modes were calculated and summed to form the single space-time SPECTRE mode \tilde{ψ}₁₀ (see Section 3.1). FIG. 12 shows three orthogonal slices of the difference in EFD power summed over all modes between conditions, averaged over all subjects. Activation in key regions of the reward circuit, including the frontal lobes, anterior cingulate gyrus, accumbens, and amygdala are clearly evident. Strong negative activation (i.e., deactivation) is evident in several structures, including the supplementary motor cortex, and the parietal operculum cortex. Activation is also apparent in the lingual gyrus and around the calcarine fissure and, as expected, in bilateral subcortical structures.

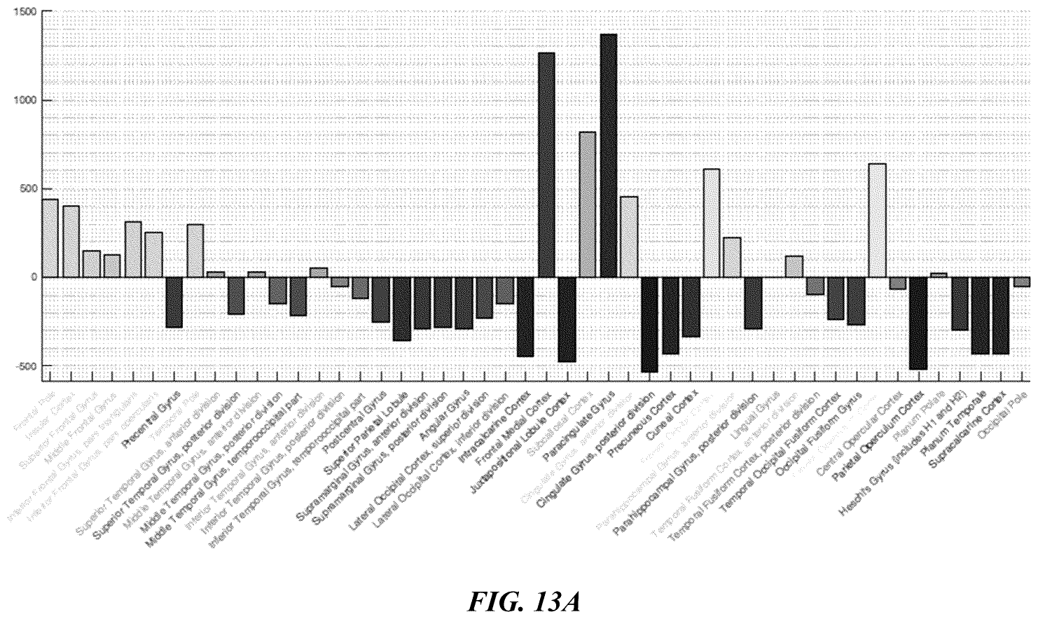

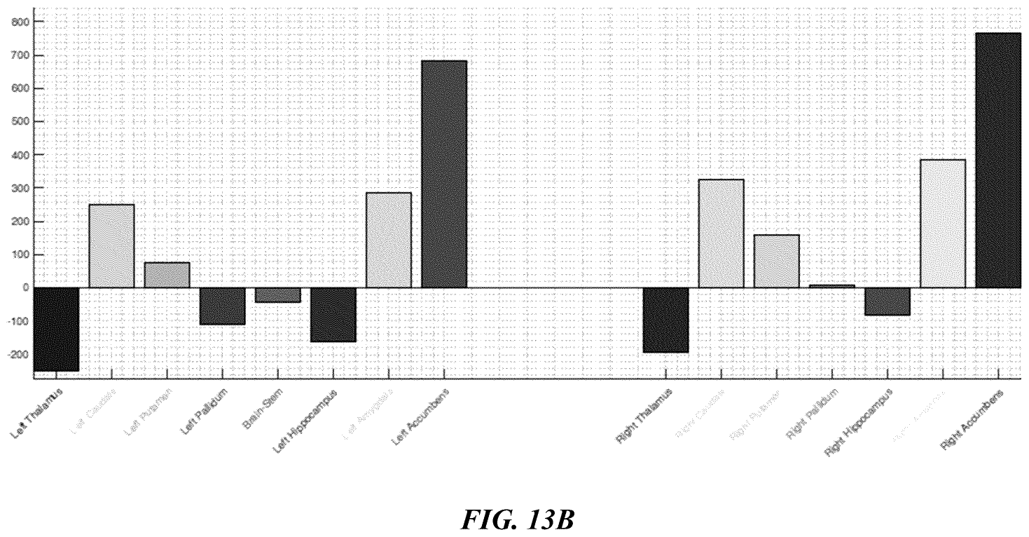

FIG. 13A shows the power per brain regions as defined by the Harvard-Oxford 2 mm cortical atlas. FIG. 13B shows the power per brain regions as defined by the Harvard-Oxford 2 mm subcortical atlas. In FIG. 13A, strong activation is apparent in the frontal cortex (medial, orbital, operculum), cingulate gyrus, paracingulate gyrus, and unsular cortex. Activation in the accumbens is apparent from the data in the subcortical atlas (FIG. 13B). These activated regions are consistent with the known elements of the human brain reward circuitry.

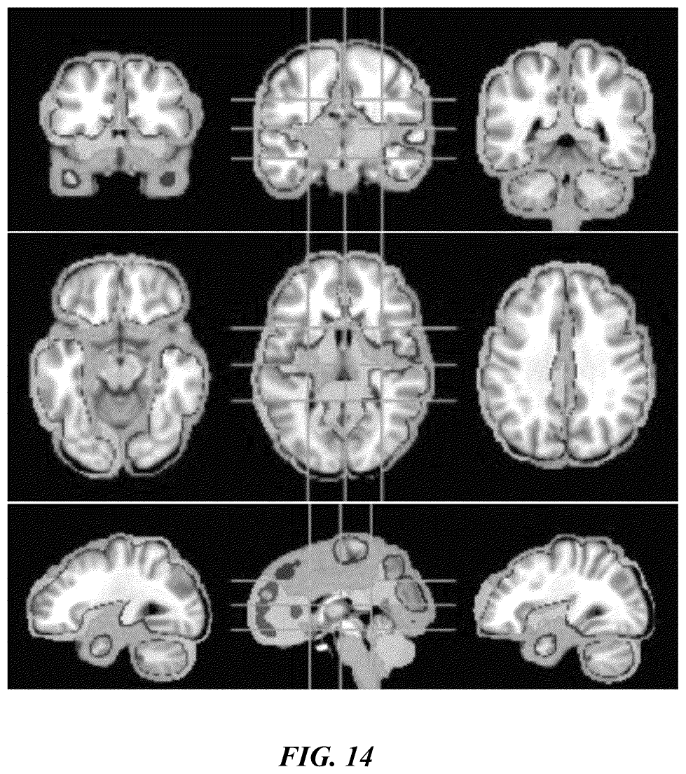

Images of statistical significance (p<0.0001) are shown in FIG. 14. It should be noted that the determination of statistical significance with SPECTRE by “traditional” methods is potentially misleading as they will tend to underestimate activation significance. The estimation of the modes in SPECTRE employs EFD which is a probabilistic formulation that by construction incorporates space-time neighborhood connectivity so that spatially and temporally coherent patterns (“clusters”) are more probable. Traditional methods have the option for “clustering” regions of activation post-hoc into their general class of techniques called “boot-strapping” or “permutation inference”. Cluster post-detection of an activation is incommensurate with our view of the estimation process wherein the clustering in space-time is a key component indicator of high-probability regions of space-time. Spatially and temporally coherent patterns may be of low amplitude with apparent low significance by traditional means, but those intensities are within a mode that contains very high significance in cortical regions (e.g., FIG. 14) which is predicted by the WETCOW framework. Thus the entire SPECTRE mode, including the somewhat diffuse lower intensity regions, is significant.

### 3.1 Example Mode Reconstruction

The reconstructed volumetric time series of the estimated electrostatic potential field can be thought of as similar to the EM equivalent of an fMRI dataset, containing a multitude of correlated spatiotemporal patterns or “modes” of the system. The problem then becomes one of detecting the multiple modes in complex non-linear systems. This problem has been addressed using EFD method, which is to be described in Section 3.2 and provides probabilistic framework for estimating spatial-temporal modes of complex non-linear systems containing multivariate interacting fields. It is formally based on a field-theoretic mathematical formulation of Bayes' Theorem that enables the hierarchy of multiple orders of field interactions including coupling between fields. Its practical utility is enabled by incorporation of the theory of entropy spectrum pathways (ESP), which uses the space-time correlations in each individual dataset to automatically select the very limited number of highly relevant field interactions. In short, it selects the configurations with maximum path entropy, summarized in the equilibrium (i.e., long time) distribution μ*.

The terminology is as follows (see Section 3.2). The k′th EFD mode of the coupled parameters α={α₁, . . . , αₘ} is denoted ψ_{α}^{(k)}(x,t) where k=1, . . . , n for some user defined n. While each of these modes provides unique information on coherent spatio-temporal activity, for characterizing the total brain activity it is often most useful and efficient to sum these modes: \tilde{ψ}ₙ=Σₖⁿψ_{α}^{(k)}(x,t). In the study, both individual modes and the summed modes are utilized, as appropriate.

A strength of the EFD method is that it uses prior information contained in individual datasets—there are no training datasets or averages across datasets—just the prior information contained within the single dataset of interest. The fact that this method uses prior information embedded within single datasets without the need for any ‘training’ is of significance to clinical studies in which important individual variations can be lost in the averaging process. It is also particularly important to the current study where validation of SPECTRE techniques necessitates comparison with single subject studies.

### 3.2 Example Entropy Field Decomposition

The entropy field decomposition (EFD) is a general probabilistic method for the estimation of spatial-temporal modes of complex non-linear, non-period, non-Gaussian multivariate data. The goal of EFD is to estimate the field ψ(x,t) that describes a continuous (in both space x and time t) parameter space from which the signal sₗ are discrete samples of sₗ=∫ψ(ξ)δ(ξ−ξₗ)dξ. In general, this can be done by constructing the posterior distribution of ψ(x,t) given the data d and any prior information I that is available, via Bayes' Theorem re-expressed in the language of field theory as

$$p ( ψ \vert s , I ) = \frac{{e}^{- H ( s , ψ )}}{Z ( s )} \qquad ( Equation 2 )$$

where Z(d)=p(d|I)=∫dψe^{-H(s,ψ)} is the partition function (considered a constant in this example) and H (s,ψ)=−ln p (s,ψ|I) is the information Hamiltonian which takes the form

$$H ( s , ψ ) = {H}_{0} - {j}^{†} ψ + \frac{1}{2} {ψ}^{†} {D}^{- 1} ψ + {H}_{i} ( s , ψ ) \qquad ( Equation 3 )$$

where H₀ is essentially a normalizing constant that can be ignored, j and D are the information source and propagator, and † means the complex conjugate transpose. Hᵢ is an interaction term

$${H}_{i} = {\sum }_{n = 1}^{∞} \frac{1}{n !} \int ⋯ \int {Λ}_{{s}_{1} ⋯ {s}_{n}}^{( n )} ψ ( {s}_{1} ) ⋯ψ ( {s}_{n} ) {ds}_{1} ⋯ {ds}_{n} \qquad ( Equation 4 )$$

where Λₛ_{₁}_{. . . s}_{ₙ}⁽ⁿ⁾ terms describe the interaction strength. In highly complex non-linear systems, j, D, and Λₛ_{₁}_{. . . s}_{ₙ}⁽ⁿ⁾ are often unknown and too complex for deriving effective and accurate approximations. In this case the ESP method, based on the principal of maximum entropy, provides a general and effective way to introduce powerful prior information to find the most significant contributions to H (d,ψ) by using coupling between different spatio-temporal points that is available from the data itself. This is accomplished by constructing a coupling matrix that characterizes the relation between locations i and j in the data Qᵢⱼ=e^{-γ}^{ᵢⱼ} where the γᵢⱼ are Lagrange multipliers that describe the relations and depend on some function of the space-time locations i and j. The eigenvalues λₖ and eigenvectors ϕ^{(k)} of the coupling matrix Q

$${\sum }_{j} {Q}_{ij} {ϕ}_{j}^{( k )} = {λ}_{k} {ϕ}_{i}^{( k )} \qquad ( Equation 5 )$$

then define the transition probability from location j to location i of the k′th mode as

$${p}_{ijk} = \frac{{Q}_{ji}}{{λ}_{k}} \frac{{ϕ}_{i}^{( k )}}{{ϕ}_{j}^{( k )}} . \qquad ( Equation 6 )$$

For each transition matrix (Equation 6) there is a unique stationary distribution associated with each path k:

$${μ}^{( k )} = {[ {ϕ}^{( k )} ]}^{2} where {μ}_{i}^{( k )} = {\sum }_{j} {μ}_{j}^{( k )} {p}_{ijk} \qquad ( Equation 7 )$$

where μ⁽¹⁾, associated with the largest eigenvalue λ₁, corresponds to the maximum entropy stationary distribution. Note that Equation 7 is written to emphasize that the squaring operation is performed on a pixel-wise basis.

The essence of the EFD approach is to incorporate these coupling matrix priors into the information Hamiltonian (Equation 3) by expanding the signal s (x,t) into a Fourier expansion using {ϕ^{(k)}} as the basis functions

$${s}_{i} = {\sum }_{k}^{K} [ {a}_{k} {ϕ}_{i}^{( k )} + {a}_{k}^{†} {ϕ}_{i}^{† , ( k )} ] \qquad ( Equation 8 )$$

which allows expressing the information Hamiltonian (Equation 3) in this ESP basis as

$$H ( d , {a}_{k} ) = - {j}_{k}^{†} {a}_{k} + \frac{1}{2} {a}_{k}^{†} Λ {a}_{k} + {\sum }_{n = 1}^{∞} \frac{1}{n !} {\sum }_{{k}_{1}}^{K} ⋯ {\sum }_{{k}_{n}}^{K} {\tilde{Λ}}_{{k}_{1} ⋯ {k}_{n}}^{( n )} {a}_{{k}_{1}} ⋯ {a}_{{k}_{n}} \qquad ( Equation 9 )$$

where matrix Λ is the diagonal matrix Diag{λ₁, . . . , λ_{K}}, composed of the eigenvalues of the coupling matrix, and jₖ=∫jϕ^{(k)}ds is the amplitude of kth mode in the expansion of the source j and the new interaction terms \tilde{Λ}⁽ⁿ⁾ are

$${\tilde{Λ}}_{{k}_{1} ⋯ {k}_{n}}^{( n )} \int ⋯ \int {Λ}_{{s}_{1} ⋯ {s}_{n}}^{( n )} {ϕ}^{( {k}_{1} )} {⋯ϕ}^{( {k}_{n} )} {ds}_{1} ⋯ {ds}_{n} \qquad ( Equation 10 )$$

from which can be derived a simple expression for the solution for the amplitudes aₖ

$$Λ {a}_{k} = {j}_{k} - {\sum }_{n = 1}^{∞} \frac{1}{n !} {\sum }_{{k}_{1}}^{K} ⋯ {\sum }_{{k}_{n}}^{K} {\tilde{Λ}}_{{kk}_{1} ⋯ {kk}_{n}}^{( n + 1 )} {a}_{{k}_{1}} ⋯ {a}_{{k}_{n}} \qquad ( Equation 11 )$$

through the eigenvalues and eigenvectors of coupling matrix.

The EFD methods can be extended to multiple modalities by incorporating coupling between different parameters, which we call Joint Estimation with Entropy Regularization (JESTER). For m=1, . . . , M different modalities d^{(m)} with the coupling matrices Q^{(m)} that all correspond to the same unknown signal S, intermodality coupling matrix can be constructed as the product of the coupling matrices for the individual modalities expressed in the ESP basis and registered to a common reference frame, which is denoted \tilde{Q}^{(m)} that is, the joint coupling matrix is [symbol] ^{(m)}=Πₘ\tilde{Q}^{(m)}. More specifically, the joint coupling matrix [symbol] ᵢⱼ between any two space-time locations (i, j) can be written in the general (equivalent) form as

$$ln {ij}_{} = {\sum }_{m = 1}^{M} {β}_{ij}^{( m )} ln {\tilde{Q}}_{ij}^{( m )} \qquad ( Equation 12 )$$

where the exponents β^{(m)} can either be some constants or functions of data collected for different modalities βᵢⱼ^{(m)} ≡β^{(m)} (\acute{d}ᵢ\tilde{d}ⱼ), \tilde{d}ᵢ≡{\tilde{d}ᵢ⁽¹⁾, . . . , \tilde{d}ᵢ^{(M)}} where \tilde{d}ᵢ^{(m)} and \tilde{Q}ᵢⱼ^{(m)} represent, respectively, the data and the coupling matrix of the modality dataset m represented in the ESP basis and evaluated at locations rᵢ and rⱼ of a common reference domain R:

$$\begin{matrix}{\tilde{d}}_{i}^{( m )} = {d}^{( m )} ( {ψ}^{( m )} ( {r}_{1} ) ) , & {\tilde{Q}}_{ij}^{( m )} = {Q}^{( m )}\end{matrix} ( {ψ}^{( m )} ( {r}_{i} ) , {ψ}^{( m )} ( {r}_{j} ) ) \qquad ( Equation 13 )$$

where ψ^{(m)}: R→X denotes a diffeomorphic mapping of m-th modality from the reference domain R to an acquisition space X.

### 3.3 Example Space-Time Information Trajectories

Space-time information trajectories are defined as streamlines generated using the global path entropy change

$${S}_{i} = - {\sum }_{k} {μ}^{( k )} {\sum }_{j} {p}_{ijk} ln {p}_{ijk} \qquad ( Equation 14 )$$

where the equilibrium μ distributions and transitional probabilities pᵢⱼ are obtained from the space-time nearest neighbor coupling matrix Qᵢⱼ^{F}(t₀)

$$Q ( {x}_{i} , {x}_{j} , {t}_{0} ) = {ij}_{m}^{} d ( {x}_{i} , {t}_{0} ) d ( {x}_{j} , {t}_{0} ) \qquad ( Equation 15 a )$$

$$Q ( {x}_{i} , {x}_{j} , {t}_{l} ) {ij}_{m}^{} ( {ϕ}^{( 1 )} ( {x}_{i} , {t}_{0} ) d ( {x}_{j} , {t}_{l} ) + {ϕ}^{( 1 )} ( {x}_{j} , {t}_{0} ) d ( {x}_{i} , {t}_{l} ) ) \qquad ( Equation 15 b )$$

where [symbol] ᵢⱼ is either the mean

$${ij}_{} = \frac{1}{T} {\int }_{0}^{T} \int ( d ( {x}_{i} , t - τ ) d ( {x}_{j} , τ ) d τ dt \qquad ( Equation 16 )$$

or the maximum

$${ij}_{} = {max}_{t} \int d ( {x}_{i} , t - τ ) d ( {x}_{j} , τ ) d τ \qquad ( Equation 17 )$$

of the temporal pair correlation function computed for spatial nearest neighbors i and j, ϕ⁽¹⁾(xⱼ,t₀) is the eigenmodes that corresponds to the largest eigenvalue of Q(xᵢ, xⱼ, t₀), and the exponent m≥0 is used to attenuate the importance of correlations. The global entropy field (Equation 14) was obtained under the Markovian assumption in the limit of long pathway lengths (or large times) and describes the global flow of information. The trajectories are generated by linearizing the Fokker-Planck equation with a potential in the form of the global entropy (Equation 14) and finding the characteristics solution using the Hamiltonian set of equations to construct the space-time trajectories are generated using geometric-optics based approach.

A goal of functional neuroimaging is to non-invasively detect and quantify the spatial and temporal variations in brain activity. Functional MRI and EEG have emerged as the most ubiquitous methods because they offer two important and complementary outcomes. Functional MRI can non-invasively detect complex spatial and relatively low frequency temporal patterns of activity related to local BOLD changes while EEG can directly detect electrical activity but without the capability of accurate spatial localization.

Ideally, one would measure the electrical activity of the brain at high temporal resolution, as done by EEG, but with the spatial localization capabilities of fMRI. However, there has been a long-standing belief that this is not possible, due to the “volume-conductance” problem. The disclosed embodiments have shown that this is not an actual physical limitation, but an artificial constraint stemming from a model for how electromagnetic wave propagate within the brain which is overly simplified by neglecting local tissue anisotropy and inhomogeneity. Implementations of the WETCOW model disclosed herein incorporate these tissue characteristics into the electromagnetic field equations (i.e., Maxwell's equations), which predicts the existence of previously undiscovered waves generated precisely as a consequence of the tissue anisotropy and inhomogeneity. Unlike standard electromagnetic waves characterized by frequencies ω that are proportional to their spatial frequency (or wavenumber) k, (i.e., ω˜k), the WETCOW waves have an inverse relationship ω˜1/k. This results in waves that can permeate throughout the brain, not necessarily along neuronal pathways, that are in precisely the spatial and temporal (frequency) range of observed brain electrical activity. This model provides the physical model upon which the SPECTRE is based, using tissue models derived from standard high resolution anatomical MRI data to reconstruct the modes of brain electrical activity throughout the entire brain volume.

Among other features and benefits, the SPECTRE reconstruction of EEG data in accordance with implementations of the disclosed technology can provide significant advantages over fMRI in temporal resolution, since EEG data has very high intrinsic temporal resolution (˜1 ms) necessary to capture rapidly varying electric field variations. Moreover, the SPECTRE algorithm can specify what frequency ranges to interrogate, providing a highly flexible analysis framework for focused investigation of particular frequency bands of interest. On the contrary, even rapid fMRI acquisition is intrinsically limited by the temporal evolution of the contrast mechanism, the BOLD signal, which is related to blood flow and thus of quite low frequency (˜1 Hz).

While the advantages of SPECTRE over fMRI in temporal resolution are clear, SPECTRE also provides advantages in spatial resolution. The inverse solution that estimates the electric field potential from the EEG data is based on a physical model of wave propagation from tissues whose composition and geometry are derived from high resolution anatomical MRI data. The final resolution of the SPECTRE electric field modes is that of the anatomical data which is typically significantly higher (˜0.5-1 mm) than the resolution of an fMRI image (˜2 mm).

It is also important to recognize that the question of resolution in fMRI is not just a question of the prescribed image resolution of the acquisition. The BOLD physical mechanism that generates the fMRI contrast is a subtle variation in the magnetic susceptibility which causes variations in the local magnetic field, that in turn alters the local signal. fMRI acquisitions are specifically designed to accentuate this effect in order to make it observable. Unfortunately, local magnetic field variations unrelated to the BOLD mechanism, in particular strong magnetic susceptibility variations due to air/tissue boundaries such as those in the sinus cavities, cause severe non-linear image distortions that effectively alter the location and shape of the affected image volume elements (voxels). This makes even the definition of ‘resolution’ problematic, as it is essentially a spatially non-linearly varying function. Such effects are absent from EEG, which is simply a set of receiving electrodes. The SPECTRE reconstruction uses high resolution MRI data acquired with techniques specifically designed to be insensitive to these magnetic susceptibility distortions and thus of very high spatial fidelity.

Some implementations of SPECTRE disclosed herein have been validated using simultaneous fMRI/EEG experiments. Example results not only affirm the capability of SPECTRE to accurately reconstruct spatial distributions of neural activity from EEG data, in alignment with the concurrently acquired fMRI data, but also reveal its efficacy in identifying robust activations across subjects that were not detectable with fMRI alone. These findings underscore the potential for disclosed embodiments based on SPECTRE to significantly enhance the sensitivity and scope of neuroimaging analyses. In some of the disclosed embodiments, further validation was performed using intra-cranial EEG measurements from an epilepsy study with reconstruction of data from a subset of sensors on the surface of the brain and were shown to be consistent with the reconstruction from all the sensors, including those directly next to the activity source. As previously described, the application of SPECTRE to high resolution EEG data during a gambling task demonstrated its ability to reconstruct a well-known and important brain circuit that has previously only been detected using fMRI. The analysis revealed significant differences in the brain networks in the alpha range 8-12 Hz, consistent with previous spatially resolved fMRI experiments but the analysis is easily carried out in any user-defined frequency ranges of interest. Disclosed embodiments based on the SPECTRE methodology are applicable to any EEG study and thus may find a wide range of applications, such as applications in ongoing neuroscience studies of reward mechanisms and in clinical applications such as addiction. Additionally, the disclosed embodiments may be implemented in applications related to neuromodulation (e.g., transcranial magnetic stimulation (TMS), transcranial focused ultrasound (tFUS), etc), drug delivery, brain computer interfaces (BCI), and monitoring of cognitive function in aging.

The implications for spatially resolved EEG are important not only from a scientific perspective, but from a practical perspective as well. Functional MRI is a much more involved and expensive procedure, requiring highly trained research or clinical applications specialists in specially designed facilities, and subjecting the subjects to a much more claustrophobic and restricted environment, with the safety concerns always present in MRI imaging experiments. On the contrary, the portability, safety, and relative ease of EEG experiments, which can be carried out in a standard research or clinical office, makes it very attractive. The high spatial and temporal resolution capabilities provided by the disclosed embodiments to standard EEG data offer the possibility of more detailed investigations of brain activity in a wide range of both basic research and clinical settings. The disclosed embodiments also have important implications for the democratization of medicine worldwide where there are many populations for which advanced technologies such as fMRI are prohibitive because of cost, citing issues for large, specialized equipment, and lack of highly trained personnel.

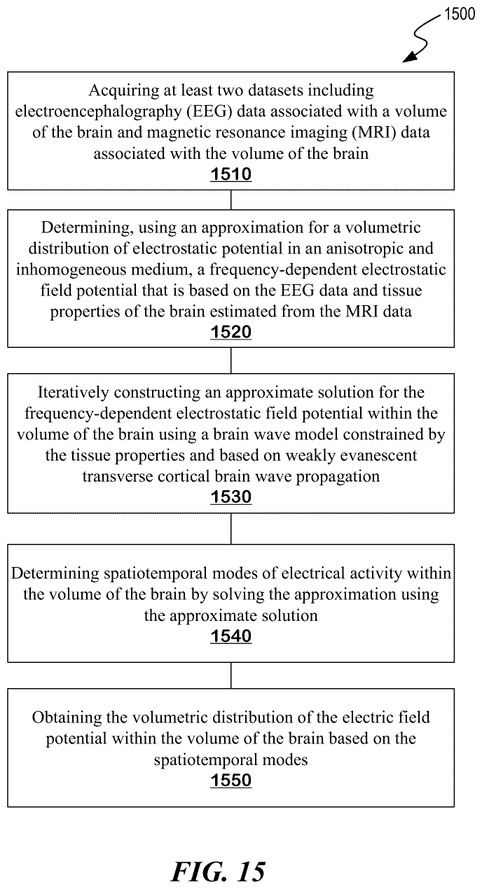

FIG. 15 shows a flow diagram of an example method 1500 that can be carried out in accordance with an example embodiment. At 1510, the method 1500 comprises acquiring at least two datasets including electroencephalography (EEG) data associated with a volume of the brain and magnetic resonance imaging (MRI) data associated with the volume of the brain. At 1520, the method 1500 comprises determining, using an approximation for a volumetric distribution of electrostatic potential in an anisotropic and inhomogeneous medium, a frequency-dependent electrostatic field potential that is based on the EEG data and tissue properties of the brain estimated from the MRI data. In some implementations, the tissue properties include morphological and electrical properties at locations within the volume of the brain. At 1530, the method 1500 comprises iteratively constructing an approximate solution for the frequency-dependent electrostatic field potential within the volume of the brain using a brain wave model constrained by the tissue properties and based on weakly evanescent transverse cortical brain wave propagation. At 1540, the method 1500 comprises determining spatiotemporal modes of electrical activity within the volume of the brain by solving the approximation using the approximate solution. At 1550, the method 1500 comprises obtaining the volumetric distribution of the electric field potential within the volume of the brain based on the spatiotemporal modes.

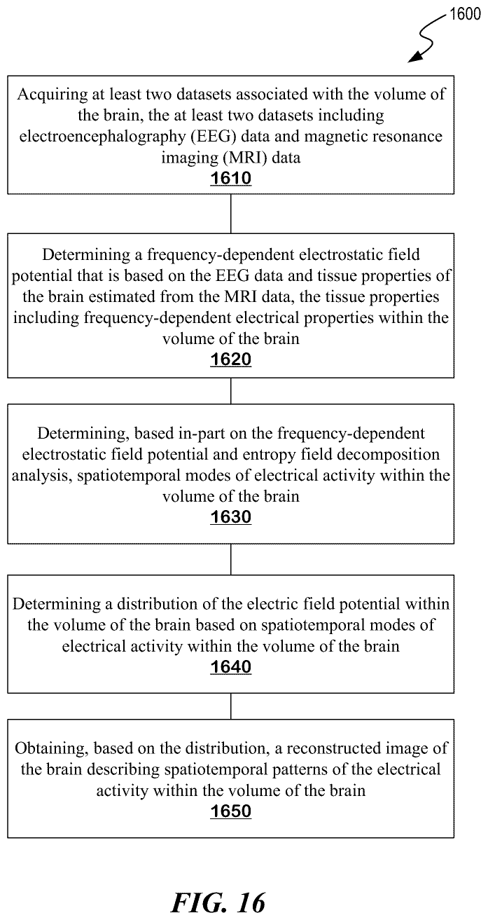

FIG. 16 shows a flow diagram of another example method 1600 that can be carried out in accordance with an example embodiment. At 1610, the method 1600 comprises acquiring at least two datasets associated with the volume of the brain, the at least two datasets including electroencephalography (EEG) data and magnetic resonance imaging (MRI) data. At 1620, the method 1600 comprises determining a frequency-dependent electrostatic field potential that is based on the EEG data and tissue properties of the brain estimated from the MRI data, the tissue properties including frequency-dependent electrical properties within the volume of the brain. At 1630, the method 1600 comprises determining, based in-part on the frequency-dependent electrostatic field potential and entropy field decomposition analysis, spatiotemporal modes of electrical activity within the volume of the brain. At 1640, the method 1600 comprises determining a distribution of the electric field potential within the volume of the brain based on spatiotemporal modes of electrical activity within the volume of the brain. At 1650, the method 1600 comprises obtaining, based on the distribution, a reconstructed image of the brain describing spatiotemporal patterns of the electrical activity within the volume of the brain.

In some example embodiments related to a method for determining volumetric distribution of electric field potential within a brain, the method comprises determining spatial and temporal patterns describing electrical activity within a volume of the brain based on spatiotemporal modes.

In some implementations of the method for determining volumetric distribution of electric field potential within the brain, at least some of the spatial and temporal patterns are used to obtain a reconstructed image of the brain.

In some implementations of the method for determining volumetric distribution of electric field potential within the brain, at least some of the spatiotemporal modes are correlated to one another.

In some implementations of the method for determining volumetric distribution of electric field potential within the brain, the volumetric distribution of the electric field potential is displayed in an image.

In some implementations of the method for determining volumetric distribution of electric field potential within the brain, at least some tissue properties of the brain are frequency-dependent.

In some implementations of the method for determining volumetric distribution of electric field potential within the brain, the spatiotemporal modes are determined using entropy field decomposition (EFD) analysis.

In some example embodiments related to a method for determining volumetric distribution of electric field potential within a brain, the method comprises determining, based in-part on entropy field decomposition (EFD) analysis, a plurality of power modes describing the electrical activity within the volume of the brain.

In some example embodiments related to a method for determining volumetric distribution of electric field potential within a brain, the method comprises: generating space-time information trajectories (STITs) based on EFD modes determined from the EFD analysis; determining, for each of the STITs, a connectivity eigenmode describing a pathway between two regions within the brain; and obtaining a reconstructed image of the brain based on the STITs and the connectivity eigenmode for each of the STITs, wherein the pathway is displayed in the reconstructed image.

In some example embodiments related to a method for determining volumetric distribution of electric field potential within a brain, the method comprises: obtaining a reconstructed image of the brain based, in-part, on at least some of the plurality of power modes, wherein regions of brain activation in the reconstructed image are associated with activity within the volume of the brain determined from EEG data.

In some example embodiments related to a method for determining volumetric distribution of electric field potential within a brain, the method comprises: summing a portion of the plurality of power modes to determine a single power mode, and obtaining a reconstructed image of the brain based, in-part, on the single power mode, wherein regions of brain activation in the reconstructed image are associated with activity within the volume of the brain determined from EEG data.

Another aspect of the disclosed embodiments relates to a processor and a memory with instructions stored thereon, wherein the instructions upon execution by the processor cause the processor to perform a method according to the technology disclosed in this patent document.

Other examples of techniques that may be implemented in accordance with the present technology are described in U.S. Patent Publication No. 10,909,414 (“Entropy field decomposition for image analysis”), U.S. Patent Publication No. 10,789,713 (“Symplectomorphic image registration”), and U.S. Patent Publication No. 11,270,445 (“Joint estimation with space-time entropy regularization”), which are incorporated by reference as part of the disclosure of this patent document for all purposes.

Various operations disclosed herein can be implemented using a processor/controller configured to include, or be coupled to, a memory that stores processor executable code that causes the processor/controller carry out various computations and processing of information. The processor/controller can further generate and transmit/receive suitable information to/from the various system components, as well as suitable input/output (IO) capabilities (e.g., wired or wireless) to transmit and receive commands and/or data. The processor/controller may be further configured to perform various method steps and computations that are disclosed in this patent document.

Various information and data processing operations described herein may be implemented in one embodiment by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), etc. Therefore, the computer-readable media that is described in the present application comprises non-transitory storage media. Generally, program modules may include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

Only a few implementations and examples are described and other implementations, enhancements and variations can be made based on what is described and illustrated in this patent document.

What is claimed:

1. A method for determining volumetric distribution of electric field potential within a brain, comprising:
acquiring at least two datasets including electroencephalography (EEG) data associated with a volume of the brain and magnetic resonance imaging (MRI) data associated with the volume of the brain;
determining, using an approximation for a volumetric distribution of electrostatic potential in an anisotropic and inhomogeneous medium, a frequency-dependent electrostatic field potential that is based on the EEG data and tissue properties of the brain estimated from the MRI data, the tissue properties including morphological and electrical properties at locations within the volume of the brain;
iteratively constructing an approximate solution for the frequency-dependent electrostatic field potential within the volume of the brain using a brain wave model constrained by the tissue properties and based on weakly evanescent transverse cortical brain wave propagation;
determining spatiotemporal modes of electrical activity within the volume of the brain by solving the approximation using the approximate solution; and
obtaining the volumetric distribution of the electric field potential within the volume of the brain based on the spatiotemporal modes.

2. The method of claim 1, further comprising:
determining spatial and temporal patterns describing the electrical activity within the volume of the brain based on the spatiotemporal modes.

3. The method of claim 2, wherein at least some of the spatial and temporal patterns are used to obtain a reconstructed image of the brain.

4. The method of claim 1, wherein at least some of the spatiotemporal modes are correlated to one another.

5. The method of claim 1, wherein the volumetric distribution of the electric field potential is displayed in an image.

6. The method of claim 1, wherein at least some of the tissue properties are frequency-dependent.

7. The method of claim 1, wherein the spatiotemporal modes are determined using entropy field decomposition analysis.

8. The method of claim 1, further comprising:
determining, based in-part on entropy field decomposition (EFD) analysis, a plurality of power modes describing the electrical activity within the volume of the brain.

9. The method of claim 8, further comprising:
generating space-time information trajectories (STITs) based on EFD modes determined from the EFD analysis;
determining, for each of the STITs, a connectivity eigenmode describing a pathway between two regions within the brain; and
obtaining a reconstructed image of the brain based on the STITs and the connectivity eigenmode for each of the STITs,
wherein the pathway is displayed in the reconstructed image.

10. The method of claim 8, further comprising:
obtaining a reconstructed image of the brain based, in-part, on at least some of the plurality of power modes,
wherein regions of brain activation in the reconstructed image are associated with activity within the volume of the brain determined from the EEG data.

11. The method of claim 8, further comprising:
summing a portion of the plurality of power modes to determine a single power mode, and
obtaining a reconstructed image of the brain based, in-part, on the single power mode,
wherein regions of brain activation in the reconstructed image are associated with activity within the volume of the brain determined from the EEG data.

12. A method for reconstructing electric field potential within a volume of a brain, comprising:
acquiring at least two datasets associated with the volume of the brain, the at least two datasets including electroencephalography (EEG) data and magnetic resonance imaging (MRI) data;
determining a frequency-dependent electrostatic field potential that is based on the EEG data and tissue properties of the brain estimated from the MRI data, the tissue properties including frequency-dependent electrical properties within the volume of the brain;
determining, based in-part on the frequency-dependent electrostatic field potential and entropy field decomposition analysis, spatiotemporal modes of electrical activity within the volume of the brain;
determining a distribution of the electric field potential within the volume of the brain based on spatiotemporal modes of electrical activity within the volume of the brain; and
obtaining, based on the distribution, a reconstructed image of the brain describing spatiotemporal patterns of the electrical activity within the volume of the brain.

13. A device, comprising:
a processor; and
a memory with instructions stored thereon, wherein the instructions upon execution by the processor cause the processor to:
acquire at least two datasets including electroencephalography (EEG) data associated with a volume of the brain and magnetic resonance imaging (MRI) data associated with the volume of the brain;
determine, using an approximation for a volumetric distribution of electrostatic potential in an anisotropic and inhomogeneous medium, a frequency-dependent electrostatic field potential that is based on the EEG data and tissue properties of the brain estimated from the MRI data, the tissue properties including morphological and electrical properties at locations within the volume of the brain;
iteratively construct an approximate solution for the frequency-dependent electrostatic field potential within the volume of the brain using a brain wave model constrained by the tissue properties and based on weakly evanescent transverse cortical brain wave propagation;
determine spatiotemporal modes of electrical activity within the volume of the brain by solving the approximation using the approximate solution; and
obtain the volumetric distribution of the electric field potential within the volume of the brain based on the spatiotemporal modes.

14. The device of claim 13, wherein the instructions upon execution by the processor further cause the processor to:
determine spatial and temporal patterns describing the electrical activity within the volume of the brain based on the spatiotemporal modes,
wherein at least some of the spatial and temporal patterns are used to obtain a reconstructed image of the brain.

15. The device of claim 13, wherein at least some of the spatiotemporal modes are correlated to one another.

16. The device of claim 13, wherein at least some of the tissue properties are frequency-dependent.

17. The device of claim 13, wherein the spatiotemporal modes are determined using entropy field decomposition analysis.

18. The device of claim 13, wherein the instructions upon execution by the processor cause the processor to:
determine, based in-part on entropy field decomposition (EFD) analysis, a plurality of power modes describing the electrical activity within the volume of the brain;
generate space-time information trajectories (STITs) based on EFD modes determined from the EFD analysis;
determine, for each of the STITs, a connectivity eigenmode describing a pathway between two regions within the brain; and
obtain a reconstructed image of the brain based on the STITs and the connectivity eigenmode for each of the STITs,
wherein the pathway is displayed in the reconstructed image.

19. The device of claim 18, wherein the instructions upon execution by the processor cause the processor to:
obtain a reconstructed image of the brain based, in-part, on at least some of the plurality of power modes,
wherein regions of brain activation in the reconstructed image are associated with activity within the volume of the brain determined from the EEG data.

20. The device of claim 18, wherein the instructions upon execution by the processor cause the processor to:
sum a portion of the plurality of power modes to determine a single power mode, and
obtain a reconstructed image of the brain based, in-part, on the single power mode,
wherein regions of brain activation in the reconstructed image are associated with activity within the volume of the brain determined from the EEG data.
